(19) United States
(12) Patent Application Publication
(10) Pub. No.: US 2005/0143300 A1
(43) Pub. Date: Jun. 30, 2005

(54) Compounds useful in inhibiting vascular leakage, inflammation and fibrosis and methods of making and using same

(76) Inventor: Jian-Xing Ma, Oklahoma City, OK (US)

Correspondence Address: DUNLAP, CODDING & ROGERS P.C., PO BOX 16370, OKLAHOMA CITY, OK, 73113, US

(21) Appl. No.: 10/963,115

(22) Filed: Oct. 12, 2004

Related U.S. Application Data
(60) Provisional application No. 60/510,620, filed on Oct. 10, 2003.

(51) Int. Cl. A61K 38/18

(52) U.S. Cl. 514/12

(57) ABSTRACT

The present invention is directed to a method of inhibiting at least one of vascular leakage, inflammation and fibrosis in an animal by administering to the animal a vascular leakage inhibiting amount of a composition, wherein at a substantially higher amount the composition is effective in inhibiting angiogenesis, and wherein the anti-angiogenic activity of the composition is separate from the vascular leakage inhibiting activity of the composition. The animal experiencing at least one of vascular leakage, inflammation and fibrosis has a disease selected from the group consisting of diabetes, chronic inflammation, brain edema, arthritis, uvietis, macular edema, cancer, hyperglycemia, a kidney inflammatory disease, a disorder resulting in kidney fibrosis, a disorder of the kidney resulting in proteinuria, and combinations thereof. The composition capable of inhibiting at least one of vascular leakage, inflammation and fibrosis is selected from the group consisting of angiostatin, fragments of angiostatin, analogs or derivatives of angiostatin, pigment epithelium-derived factor, fragments of pigment epithelium-derived factor, analogs or derivatives of pigment epithelium-derived factor and combinations thereof.

### CROSS REFERENCE TO RELATED APPLICATIONS

This application claims benefit under 35 U.S.C. 119(e) of provisional application U.S. Ser. No. 60/510,620, filed Oct. 10, 2003, the contents of which are hereby expressly incorporated herein by reference.

### STATEMENT REGARDING FEDERALLY SPONSORED RESEARCH OR DEVELOPMENT

The U.S. government may own or have rights in and to this invention pursuant to NIH grant Nos. EY12600 and EY015650.

### BACKGROUND OF THE INVENTION

1. Field of the Invention

The present invention relates, in general, to compounds useful for inhibiting at least one of vascular leakage, inflammation and fibrosis and methods of making and using same. More particularly, but not by way of limitation, the present invention relates to compounds that are capable of blocking at least one of vascular leakage, inflammation and fibrosis in patients (broadly, an animal and more particularly, a mammal or human) that have pathologic vascular leakage, inflammation and fibrosis. Although not to be regarded as limiting, the compounds disclosed herein and their methods of use are particularly useful in inhibiting at least one of vascular leakage, inflammation and fibrosis in the retina and kidney.

2. Background of the Invention

Breakdown of the blood-retinal barrier (BRB), increased vascular permeability and vascular leakage are early complications of diabetes and a major cause of diabetic macular edema (Cunha-Vaz et al., 1985; and Yoshida et al., 1993). At early stages of diabetic retinopathy, it has been determined that the increase of retinal vascular permeability precedes the appearance of clinical retinopathy (Cunha-Vaz et al., 1985; and Yoshida et al., 1993). As there is no satisfactory, non-invasive therapy, diabetic macular edema is a major cause of vision loss in diabetic patients (Moss et al., 1998). Although the pathogenic mechanism underlying the breakdown of the blood-retinal barrier and the increase of retinal vascular permeability is uncertain, the over-production of VEGF (Vascular Endothelial Growth Factor) in the retina is believed to play a key role in the development of vascular hyper-permeability in diabetes (Murata et al., 1996; and Hammes et al., 1998).

VEGF is also referred to as vascular permeability factor (VPF) based on its potent ability to increase vascular permeability (Dvorak et al., 1995; and Aiello et al., 1997). It has been identified as a major causative factor in retinal vascular hyper-permeability (Aiello et al., 1997). The over-expression of VEGF or its receptors is associated with an increased vascular permeability in the retina of streptozotocin (STZ)-induced diabetes (Qaum et al., 2001). There are two possible mechanisms responsible for VEGF-induced vascular hyper-permeability. First, VEGF may act directly on the tight junction of endothelial cells, as it has been shown that VEGF alters the tight junction proteins such as the phosphorylation of occludin and ZO-1 (Antonetti et al., 1999). Second, VEGF may act through the leukocyte-endothelial cell interaction which can trigger endothelial cell adherence and tight junction disorganization (Del Maschio et al., 1996; and Bolton et al., 1998). VEGF has been shown to increase leukocyte stasis through the up-regulation of intercellular adhesion molecule-1 (ICAM-1) (Miyamoto et al., 2000), suggesting that VEGF is also an inflammatory factor. Over-production of VEGF in diabetic retina is believed to be the major cause of vascular leakage, leukostasis and retinal edema, as well as retinal neovascularization in diabetic retinopathy (Aiello et al., 2000).

Diabetic nephropathy (DN) is another one of the most important microvascular complications of diabetes, and DN occurs in 30-40% of diabetic patients (Raptis et al., 2001; and American Diabetes Assoc., 2000). The early changes in DN are characterized by thickening of the glomerular basement membrane and expanded extracellular matrix (ECM), leading to glomerular hyper-filtration and microalbuminuria, renal inflammation and glomerular fibrosis (Raptis et al., 2001; and Sakharova et al., 2001). Although intensified control of hyperglycemia, blood pressure and hyperlipidemia reduces the risks of DN, it does not sufficiently prevent diabetic patients with microalbuminuria from progressing to devastating overt DN, a leading cause of end-stage renal diseases (American Diabetes Assoc., 2000; Anonymous, 1995; and Anonymous, 2000). The exact pathogenesis of DN remains largely unknown.

As with diabetic retinopathy, several growth factors have been suggested to be involved in the pathogenesis of DN, most importantly, transforming growth factor-β (TGF-β) and vascular endothelial growth factor (VEGF) (Chiarelli et al., 2000; and Cooper et al., 2001). TGF-δ has been recognized as a modulator of ECM formation. Over-expression of TGF-δ in diabetic glomeruli is believed to contribute to matrix accumulation by increasing synthesis and decreasing degradation of extracellular proteins such as fibronectin, leading to glomerular fibrosis (Goldfarb et al., 2001; Greener, 2000; Ng et al., 2003; and Tamaki et al., 2003). Accumulating evidence indicates that VEGF and TGF-β are key pathogenic factors in early stages of DN (Iglesias-de la Cruz et al., 2002; Gambaro et al., 2000; Lane et al., 2001; Kim et al., 2003; Senthil et al., 2003; and Bortoloso et al., 2001). Serum and urinary TGF-β levels have been found to correlate with the severity of microalbuminuri (Pfeiffer et al., 1996; and Ellis et al., 1998). Therefore the increase of the systemic TGF-β levels has been suggested as a marker for DN (Mogyorosi et al., 2000).

Angiostatin is a proteolytic fragment (kringle 1-4) of plasminogen (O'Reilly et al., 1994). It was identified as a potent angiogenic inhibitor which blocks neovascularization and suppresses tumor growth and metastases. Angiostatin specifically inhibits proliferation and induces apoptosis in vascular endothelial cells (Claesson et al., 1998). Recent evidence has suggested that decreased angiostatin levels in the vitreous may play a role in the development of proliferative diabetic retinopathy (Spranger et al., 2000). Moreover, recombinant angiostatin has been shown to block retinal neovascularization in a rat model of oxygen-induced retinopathy (OIR) (Meneses et al., 2001). Delivery of a recombinant virus expressing angiostatin has been found to suppress laser-induced choroidal neovascularization (Lai et al., 2001). These findings reveal therapeutic potential of angiostatin in the treatment of retinal neovascularization as well as in the treatment of cancer.

The mechanism responsible for the anti-angiogenic activity of angiostatin is currently uncertain. However, angiostatin has been found to inhibit VEGF- and bFGF-induced activation of p42/p44 MAP kinase (Anonymous, 2000). As VEGF- and bFGF-induced angiogenesis is mediated, in part, through the MAP kinase pathway, blocking the activation of MAP kinase has been suggested to be a possible mechanism responsible for the anti-angiogenic activity of angiostatin (Flyvhjerg, 2000; and Chen et al., 2003). Recent evidence has shown that angiostatin binds to integrins, predominantly αᵥβ₃, on the surface of endothelial cells, but does not induce stress fiber formation, implying that the anti-angiogenic activity of angiostatin may be through interfering with the αᵥβ₃-mediated signaling in endothelial cells (Goldfarb et al., 2001).

Pigment epithelium-derived factor (PEDF) is a multi-functional serine proteinase inhibitor (Tombran-Tink et al., 1995). Although PEDF was first identified in the eye, it is widely distributed in a variety of organs, such as brain, spinal cord, liver, heart, placenta, bone, pancreas and prostate (Karakousis et al., 2001; Tombran-Tink et al., 2003a; and Tombran-Tink et al., 2003b). As a potent anti-angiogenic factor, PEDF plays an important role in maintaining the homeostasis of the ocular vascular system (Tombran-Tink et al., 2003a; Tombran-Tink et al., 2003b; and Dawson et al., 1999). A recent study has shown that PEDF also reduces VEGF-induced vascular hyper-permeability (Liu et al., 2004). In diabetic retinopathy, decreased endogenous PEDF and increased VEGF levels break the delicate balance between angiogenic stimulators and inhibitors, resulting in retinal vascular hyper-permeability and retinal neovascularization (Boehm et al., 2003; Gao et al., 2001; and Gao et al., 2002). Systemic injection of PEDF or virus-mediated delivery of PEDF significantly inhibits retinal angiogenesis (Mori et al., 2001; and Stellmach et al., 2001). Recent studies demonstrated that down-regulation of PEDF may also contribute to the development of renal and prostate cancer (Doll et al., 2003; and Abramson et al., 2003).

There is currently a need in the art for new methods of specifically inhibiting vascular leakage, inflammation and fibrosis that are effective and substantially non-toxic to the animal suffering from pathologic vascular leakage, inflammation and fibrosis. It is to such methods that the presently disclosed and enabled invention are directed.

### SUMMARY OF THE INVENTION

According to the present invention, methods of inhibiting at least one of vascular leakage, inflammation and fibrosis are provided. Broadly, the present invention is related to a new function that has been discovered for a group of proteins and peptides that are known to function as inhibitors of angiogenesis. The methods of the present invention involve administration of a composition previously identified to have anti-angiogenic properties, wherein the vascular leakage inhibiting activity of the composition is separate from and occurs at a much lower dosage than the dosage at which the anti-angiogenic activity of the composition occurs.

It is an object of the present invention to provide a method of inhibiting at least one of vascular leakage, inflammation and fibrosis in an animal (such as a mammal or human) suffering from pathologic vascular leakage, inflammation and fibrosis or having a predisposition for vascular leakage, inflammation and/or fibrosis. The method includes administering to the animal a vascular leakage inhibiting amount of a composition, wherein at a substantially higher amount the composition is effective in inhibiting angiogenesis, and wherein the anti-angiogenic activity of the composition is separate from the vascular leakage inhibiting activity of the composition. The animal experiencing vascular leakage may have a disease (or be predisposed to a disease) selected from the group consisting of diabetes, chronic inflammation, brain edema, edema, arthritis, uvietis, ascites, macular edema, cancer, hyperglycemia, a kidney inflammatory disease, a disorder resulting in kidney fibrosis, a disorder of the kidney resulting in proteinuria, and combinations thereof. The compositions of the present invention not only inhibit vascular leakage but also inhibit chronic inflammation as found in the diabetic retina and kidney and also prevent fibrosis in these tissues.

It is another object of the present invention, while achieving the before-stated object, to provide a method of inhibiting at least one of vascular leakage, inflammation and fibrosis in an animal by administering an effective amount of a composition capable of inhibiting at least one of vascular leakage, inflammation and fibrosis to an animal, in need thereof, wherein the composition capable of inhibiting at least one of vascular leakage, inflammation and fibrosis is selected from the group consisting of angiostatin, fragments of angiostatin, analogs or derivatives of angiostatin, pigment epithelium-derived factor, fragments of pigment epithelium-derived factor, analogs or derivatives of pigment epithelium-derived factor, SLED compounds, and combinations thereof.

It is a further object of the present invention, while achieving the before-stated object, to provide a composition having an activity that inhibits at least one of vascular leakage, inflammation and fibrosis and an activity that inhibits angiogenesis, wherein a substantially higher amount of the composition must be administered to an animal for the composition to exhibit the inhibition of angiogenesis activity, whereas a substantially lower amount of the composition exhibits the activity that inhibits at least one of vascular leakage, inflammation and fibrosis when administered to an animal.

The amount of the composition required to exhibit the activity of inhibiting of at least one of vascular leakage, inflammation and fibrosis in an animal may be at least 10-fold lower than the amount required to exhibit the anti-angiogenic activity of the composition, and preferably may be at least 50-fold lower than the amount required to exhibit the anti-angiogenic activity of the composition, and more preferably may be at least 100-fold lower than the amount required to exhibit the anti-angiogenic activity of the composition.

The vascular leakage inhibiting amount of the composition utilized in accordance with the methods of the present invention may substantially decrease the overexpression of VEGF or TGF-β in the retinas and/or kidneys of the animal, or may substantially decrease extracellular matrix production in the kidneys of the animal, or may substantially decrease overexpression of at least one inflammatory factor, such as MCP-1, in the affected organs or tissues of the animal.

The compositions utilized in accordance with the present invention are preferably naturally occurring proteins or peptides that exhibit substantially no toxicity in the animal.

Other objects, features and advantages of the present invention will become apparent from the following detailed description when read in conjunction with the accompanying drawings and appended claims.

### BRIEF DESCRIPTION OF THE SEVERAL VIEWS OF THE DRAWINGS

FIG. 1 illustrates the dose-dependent reduction of vascular permeability in OIR rat retina by angiostatin. Rats received an intravitreal injection of angiostatin in the right eye and PBS in the left eye at age P14. Retinal vascular permeability was measured at P16 using the Evans blue method and normalized by total protein concentrations in the retina. Permeability was expressed as a percentage of the contralateral control (mean±SD, n=4). 1, age-matched normal rats with a PBS injection; 2, OIR rats with a PBS injection; 3, 4 and 5, OIR rats with an injection of 1.875, 3.75 and 7.5 μg/eye of angiostatin, respectively.

FIG. 2 illustrates a time course of the angiostatin-induced reduction of vascular permeability in OIR rats. At age P14, the right eye of OIR rats received an intravitreal injection of angiostatin (7.5 μg/eye), and the left eye of OIR rats received an intravitreal injection of the same volume of PBS as the control. Vascular permeability was measured at one, two and three days after injection. Vascular permeability was normalized by the total protein concentrations in the retina and expressed as a percentage of the contralateral control (mean±SD, n=4). ns, not statistically significant.

FIG. 3 illustrates angiostatin-induced reduction of vascular permeability in STZ-diabetic rats. Angiostatin was injected into the vitreous of the right eye (7.5 μg/eye) and PBS into the left eye of STZ-diabetic rats (A, B) and normal adult rats (C, D). Vascular permeability in the retina and iris was measured 2 days after the injection, normalized by total protein concentrations in the tissues and expressed as a percentage of the contralateral control (mean±SD, n=4). (A, B) Angiostatin reduced vascular permeability in the retina and iris of STZ-diabetic rats. (C, D) Angiostatin does not affect vascular permeability in normal rats. PBS, PBS-injected eye; Ang, angiostatin-injected eye.

FIG. 4 illustrates angiostatin-mediated down-regulation of VEGF expression in retinas of OIR and STZ-diabetic rats. OIR rats (P14), STZ-diabetic rats (2 weeks of diabetes) and normal adult rats received an intravitreal injection of angiostatin (7.5 μg/eye) in the right eye and PBS in the left eye. Retinal VEGF levels were determined by Western blot analysis using an anti-VEGF antibody one day after the injection. The same membranes were stripped and re-blotted with anti-β-actin antibody (A). Each blot is a representative of the results from 3 rats in each group. VEGF levels were semi-quantified by densitometry, normalized by β-actin levels and expressed as a percentage of control (B). PBS, PBS-injected eye; Ang, angiostatin-injected eye; ns, not statistically significant.

FIG. 5 illustrates the immunohistochemistry of VEGF in eyes of normal, OIR and angiostatin-treated OIR rats. Rats with OIR received an intravitreal injection of 7.5 μg/eye angiostatin in the right eye and PBS in the left eye at age P14. The eye was enucleated at P15, and retinal sections were labeled with an anti-VEGF antibody using the ABC method. VEGF signal is shown in brown color. (A) retina from the OIR rat with PBS injection stained in the absence of the anti-VEGF antibody for negative control; (B) retina from the OIR rat after PBS injection; (C) retina from OIR rat after angiostatin injection. GCL, ganglion cell layer; INL, inner nuclear layer; ONL, outer nuclear layer; RPE, retinal pigment epithelium.

FIG. 6 illustrates a Western blot analysis of angiostatin in the kidney of normal and diabetic BN rats. (A) Western blot analysis of angiostatin in the kidney, liver and retina of normal adult BN rats. (B) Western blot analysis of angiostatin in the kidney of 6-week diabetic rats and age-matched normal controls. Equal amounts (50 μg) of total protein from each sample were blotted with a specific anti-angiostatin antibody, which recognized angiostatin as well as its parent protein plasminogen and plasmin. The results showed that angiostatin existed in two forms at the molecular weight of 50 kDa and 38 kDa in high amounts in the normal rat kidney. The angiostatin level was dramatically decreased in the kidney of 6-week diabetic rats when compared with that in the age-matched controls.

FIG. 7 illustrates a decrease in angiostatin levels and MMP-2 expression in the diabetic cortex and medulla. (A) Western blot analysis of angiostatin, ICAM-1, and VEGF in the cortex and medulla of 6-week diabetic BN rats and age-matched controls: the cortex and medulla were carefully dissected from diabetic and control rats. 50 μg of total protein from each sample was blotted with a specific anti-angiostatin antibody. The same membrane was stripped and re-blotted with an antibody specific to rat ICAM-1 and VEGF. The results showed that the angiostatin level was dramatically decreased in diabetic cortex and medulla while the ICAM-1 and VEGF levels were significantly increased. (B) Quantification of the MMP-2 mRNA in the cortex and medulla by real-time RT-PCR: total RNA was isolated from the tissue, and the mRNA level of MMP-2 was determined by real-time RT-PCR and normalized by 18s RNA levels. The average mRNA level was expressed as a percentage of control (mean±SD, n=4). Grey bars represent normal rats, and the black bars represent diabetic rats. (C) MMP-2 activity analysis by zymography: 15 μg of tissue extracts were applied to a pre-cast 10% polyacrylamide gel copolymerized with 1 mg/ml gelatin. After electrophoresis, the gel was renatured and developed. A clear band was observed at 66 kDa representing that area digested by active MMP-2.

FIG. 8 illustrates angiostatin-induced down-regulation of VEGF and TGF-β expression in HMC. HMC were incubated with high glucose (30 mM) in the absence or presence of 100 nM angiostatin, and mannitol was used as the osmolarity control. The medium was harvested at 48 h, 72 h, and 96 h after incubation. VEGF levels (A) and TGF-β levels (B) in the medium were measured by ELISA. The results were normalized by total protein concentration and expressed as pg per mg total protein in the medium (mean±SD, n=3). Values statistically different from the normal glucose controls are indicated by *P<0.05, **P<0.01. Values statistically different from the high glucose are indicated by †P<0.05, ‡P<0.01.

FIG. 9 illustrates angiostatin-induced decrease of MCP-1, a major inflammatory factor, secretion in HMC. HMC were incubated with high glucose at a concentration of 30 mM (A) or 5 ng/ml of TGF-β (B) in the absence or presence of different doses of angiostatin (0.4-250 nM) for 48 h. MCP-1 levels in the medium were measured by ELISA. The results were normalized by total protein concentration and expressed as ng per mg total protein in the medium (mean±SD, n=3). Values statistically different from the normal glucose controls are indicated by **P<0.01. Values statistically different from the high glucose or TGF-β treatments are indicated by †P<0.05, ‡P<0.01.

FIG. 10 illustrates angiostatin-induced up-regulation of PEDF expression in HMC. Cultured human mesangial cells were incubated with 5 ng/ml of TGF-β (A) or 50 ng/ml of angiotensin II (B) in the absence or presence of different doses of angiostatin (0.4-250 nM) for 48 h. PEDF levels in the medium were measured by ELISA. The results were normalized by total protein concentration and expressed as ng per mg total protein in the medium (mean±SD, n=3). Values statistically different from the controls are indicated by *P<0.05, **P<0.01. Values statistically different from TGF-β or angiotensin II-treatment are indicated by †P<0.05, ‡P<0.01.

FIG. 11 illustrates angiostatin-induced decrease of fibronectin production in HMC. HMC were incubated with high glucose 30 mM (A) or 50 ng/ml of angiotensin II (B) in the absence or presence of different doses of angiostatin (0.4-250 nM) for 48 h. The fibronectin levels in the medium were measured by ELISA. The results were normalized by total protein concentration and expressed as ng per mg total protein in the medium (mean±SD, n=3). Values statistically different from the normal glucose controls are indicated by **P<0.01. Values statistically different from high glucose or angiotensin II-treated are indicated by ‡P<0.01.

FIG. 12 illustrates angiostatin-induced inhibition of TGF-β-induced fibronectin overproduction by blockade of Smad-2/3 activation in HMC. (A) HMC were incubated with 5 ng/ml of TGF-β in the absence or presence of different doses of angiostatin (0.4-250 nM) for 48 h. Fibronectin levels in the medium were measured by ELISA. The results were normalized by total protein concentration and expressed as ng per mg total protein in the medium (mean±SD, n=3). Values statistically different from the controls are indicated by **P<0.01, Values statistically different from TGF-β-treated are indicated by †P<0.05, ‡P<0.01. (B) HMC were incubated with 5 ng/ml of TGF-β in the absence or presence of 100 nM angiostatin for 1 h. The cells were fixed and stained by anti-Smad-2/3 antibody and visualized under fluorescein microscope. Significant increase of Smad-2/3 expression and nuclear translocation was observed in the cells exposed to TGF-β (FIG. 12B-b, 400×), when compared to that in the control cells (FIG. 12B-a, 400×). 100 nM of Angiostatin effectively blocked the up-regulation and translocation of Smad-2/3 (FIG. 12B-c, 400×).

FIG. 13 illustrates that angiostatin had no effect on cell proliferation in HMC. The tetrazolium dye-reduction (MTT) assay (Sigma, Mich.) was used to determine the number of viable human mesangial cells after treatments with different doses of angiostatin for 3 days under normal glucose (A) and high glucose (B) conditions. The results showed that angiostatin had no effect on cell viability in HMC.

FIG. 14 illustrates a quantitative comparison of PEDF levels in the kidney, liver and retina of normal rats. (A) Western blot analysis of PEDF: tissue samples were obtained from 9-week-old BN rats. Equal amounts (50 μg) of total protein from each sample were blotted with a specific anti-PEDF antibody. The same membrane was stripped and re-blotted with an antibody specific to β-actin. (B) Quantitative analysis of PEDF by ELISA: PEDF was quantified by ELISA and normalized by total protein concentration. Average PEDF levels were expressed as ng per mg of total protein in the tissue (mean±SD, n=6).

FIG. 15 illustrates the localization of PEDF in the renal tissues. (A) Western blot analysis of PEDF: the cortex and medulla were carefully dissected from 9-week-old BN rats. 5.0 pg of total protein from each sample was blotted with a specific anti-PEDF antibody. The same membrane was stripped and re-blotted with an antibody specific to β-actin. (B) Quantitative analysis of PEDF by ELISA: the protein levels of PEDF were quantified by ELISA and normalized by total protein concentration. Average PEDF levels were expressed as ng per mg of protein in the tissue (mean±SD, n=6). (C) Immunohistochemistry of PEDF in normal rat kidney. In the cortex (C-a, 200× and C-b, 600×), PEDF signal was mainly detected in the glomeruli along the parietal glomerular capsule and basement membrane. In the medulla (C-c, 200×), PEDF signal was observed at the tubular basement membrane and interstitial tissue, but much weaker than that in the glomeruli. (D) Immunohistochemistry in normal rat glomeruli illustrates the similar but not identical patterns of PEDF (D-a, 400×) and synaptopodin (D-b, 400×) signals. The arrowhead indicates the PEDF signals at the parietal capsule of glomeruli.

FIG. 16 illustrates decreased expression of PEDF in the kidney of diabetic rats. (A) Western blot analysis of PEDF in the kidney: equal amounts (50 μg) of kidney proteins from diabetic rats and normal controls were used for PEDF Western blot analysis. (B) Quantitative analysis of PEDF in the cortex and medulla by ELISA: the cortex and medulla were dissected from the kidneys of rats of 6-week diabetes and age-matched normal controls. PEDF protein in the tissue extract was quantified by ELISA and normalized by the total protein concentration. PEDF levels were expressed as ng per mg of protein in the tissue (mean±SD, n=4). (C) Quantification of PEDF mRNA in the cortex and medulla by real-time RT-PCR: total RNA was isolated from the tissue, and the mRNA level of PEDF was determined by real-time RT-PCR and normalized by 18s RNA levels. The average mRNA level was expressed as a percentage of control (mean±SD, n=4). (D) The protein levels of TGF-β and (E) protein levels of fibronectin in kidney extracts from rats of 6-week diabetes and age-matched controls were quantified by specific ELISA and normalized by the total protein (mean±SD, n=4).

FIG. 17 illustrates immunohistochemistry analysis of PEDF in the kidneys of diabetic rats. (A) Immunostaining for PEDF in 9-week-old normal rat kidneys (A-b) and diabetic rat kidneys after diabetes onset for 2 weeks (A-d) and 4 weeks (A-f). The panels A-a, A-c and A-e represented phase contrast for the same field of A-b, A-d and A-f, respectively. Magnification: 100×. Arrows indicate the locations of glomeruli. (B) Immunostaining for PEDF in the kidney of an 8-month-old normal rat (B-a, B-b) and age-matched diabetic rats 6 months after the onset of diabetes (B-c, B-d). (B-a, B-c), the glomeruli (600×); (B-b, B-d), the medulla (400×). The results showed that in the normal rat cortex, PEDF was highly expressed in the glomeruli (A-b). In diabetic rat kidney, dramatic decreases of PEDF expression were observed in the glomeruli (A-d, A-f, B-c), but not in the medulla (B-d). (C) Western blot analysis of PEDF in isolated glomeruli from rats with 8-week diabetes and age-matched non-diabetic controls. Fifty μg of total protein from each sample were blotted with a specific anti-PEDF antibody. The same membrane was stripped and re-blotted with an antibody specific to β-actin. (D) Immunohistochemistry of PEDF and synaptopodin in the kidneys of 2-week diabetes and non-diabetic controls. (D-a, D-c), PEDF staining; (D-b, D-d), synaptopodin staining. Compared with the staining in normal kidneys (D-a, D-b), the PEDF expression was dramatically decreased (D-c), while no detectable podocyte loss (D-d) was observed in the same diabetic kidney (200×).

FIG. 18 illustrates high glucose-induced decrease of PEDF secretion in cultured HMC. HMC were treated with different concentrations of D-glucose and D-mannitol (osmotic control) for 96 h. The medium was harvested by centrifugation, and PEDF in the supernatant was measured by PEDF ELISA. All the assays were run in triplicate, and PEDF concentration was normalized by the total protein in the medium. The results were expressed as ng PEDF per mg total protein in the medium (mean±SD). Values statistically different from the normal glucose controls are indicated by *P<0.05, **P<0.01.

FIG. 19 illustrates PEDF-induced down-regulation of TGF-β expression in HMC. (A) HMC were incubated with high glucose (30 mM) for 48 h; then PEDF was added at different concentrations (2.5-160 nM) and incubated for another 48 h. TGF-β in the medium was measured by ELISA. (B) After incubation with high glucose (30 mM) for 48 h, 40 nM PEDF was added to the medium of HMC. The medium was harvested at 24 h and 48 h after the addition of PEDF. The TGF-β concentrations in the medium were determined by ELISA. The results were expressed as μg per mg total protein in the medium (mean±SD).

FIG. 20 illustrates PEDF-induced decrease of fibronectin secretion from HMC. (A) After incubation of HMC with high glucose (30 mM) for 48 h, PEDF was added to HMC cultures at different concentrations (5-40 nM) and incubated for another 48 h. The medium was collected for fibronectin ELISA. (B) After incubation of HMC with 30 M glucose for 48 h, 40 nM PEDF was added to the culture medium. The medium was harvested at 24 h and 48 h after the addition of PEDF. Fibronectin levels in the medium were determined by ELISA. The results were expressed as μg per mg of total proteins in the medium (mean±SD). Values statistically different from the cells cultured in 30 mM glucose without PEDF are indicated by *P<0.05, **P<0.01.

FIG. 21 illustrates PEDF dose-dependent reduction of vascular leakage in OIR rats. PEDF was injected intravitreally (3 μl of solution with PEDF concentration as indicated) into the right eye of OIR rats at the dose as indicated and PBS into the left eye as the control at P14. For comparison, PBS was also injected into the age-matched normal rats. Vascular permeability was measured at P16. The permeability was normalized by total protein concentration in the tissues and expressed as percentages of respective controls (mean±SD, n=4). The average value of each dose group was compared with the respective control using paired Student's t-test, and P values are provided. This result demonstrates that PEDF at a dose as low as 0.375 μg/eye (3 μl of 0.125 μg/μl) can significantly reduce vascular permeability.

FIG. 22 illustrates a time course of the effect of PEDF on vascular permeability. PEDF was injected into the vitreous of the right eye of OIR rats at P14 (3 μg/eye) and PBS into the left eye as the control. Vascular permeability was measured at P15, P16, P17 and P18. Vascular permeability was normalized by protein concentrations and expressed as percentages of respective controls (mean±SD, n=4). The value at each time point was compared with the respective control using paired Student's t-test, and P values are provided. The results demonstrate that PEDF reduces vascular permeability at 1 and 2 days after the injection. By days three and four, the protein is degraded, which correlates with the diminished effect on permeability.

FIG. 23 ilustrates PEDF-induced reduction of vascular leakage in STZ-induced diabetes. STZ-diabetic Brown Norway rats received an intravitreal injection of 3 μg/eye PEDF at two weeks after induction of diabetes. Vascular permeability was measured in the retina, iris and choroid two days after injection and normalized by protein concentrations (mean±SD, n=4). Significant difference in permeability was observed only in the retina, but not in the iris. This result indicates that PEDF also reduces vascular leakage in the STZ-diabetic model.

FIG. 24 demonstrates that PEDF competes with VEGF for binding to RCEC. ¹²⁵I-VEGF was incubated with primary RCEC in the absence or presence of various concentrations of PEDF or K5 for 2 hr. After the unbound ¹²⁵I-VEGF was washed off, the VEGF bound to the cells was quantified by a γ counter and converted to fmoles based on VEGF standard. Values are mean±SD (n=3).

FIG. 25 illustrates PEDF-induced down-regulation of VEGF expression in RCEC and in the retina. (A) PEDF decreased VEGF expression in RCEC. RCEC were treated with various concentrations of PEDF as indicated under hypoxia for 24 h. VEGF levels in cell lysates were measured by Western blot analysis using an anti-VEGF antibody. The same blot was stripped and reblotted with an anti-β-actin antibody. (B) PEDF-induced down-regulation of VEGF in the retina of OIR rats. The OIR rats received an intravitreal injection of 3 μg PEDF in the right eye and the same volume of PBS in the left. The retinas from 3 OIR rats were pooled for Western blot analysis of VEGF and β-actin levels. The results showed that PEDF down-regulates VEGF expression, which may be responsible for the effect on vascular leakage.

FIG. 26 illustrates that PEDF decreases the expression of MCP-1 in the retina of STZ-diabetic rats and rats with OIR. PEDF was injected into the vitreous of the right eye of rats with 6 weeks of diabetes and rats with OIR at P16. The left eye received the same volume PBS as control. MCP-1 levels in the retina were measured by ELISA and normalized by total protein concentration. The results showed that PEDF significantly decreases MCP-1 levels (P<0.01, n=4), demonstrating that PEDF inhibits inflammation induced by diabetes and ischemia.

FIG. 27 demonstrates that adenovirus-mediated PEDF gene delivery reduces albuminuria in diabetic rats. Diabetes was induced by injection of STZ and confirmed by blood glucose measurement. Two weeks following the onset of diabetes, the diabetic rats were randomly assigned into control and treatment groups. Rats in the treatment group received an intraperitoneal injection of adenovirus expressing PEDF, and the control group received an intraperitoneal injection of an adenovirus without the PEDF gene. The 24-h urine was collected individually at one, two three and four weeks after the viral injection. Albumin and creatinine concentrations in the urine were measured using commercial kits. Albumin levels were normalized by creatinine and compared to that of the control group. The results showed that PEDF significantly reduced albuminuria in diabetic rats at two, three and four weeks after the gene delivery (P<0.05, n=4).

### DETAILED DESCRIPTION OF THE INVENTION

Before explaining at least one embodiment of the invention in detail, it is to be understood that the invention is not limited in its application to the details of construction and the arrangement of the components set forth in the following description, the experimental details or results, or illustrated in the appended drawings. The invention is capable of other embodiments or of being practiced or carried out in various ways that would be appreciated by one of ordinary skill in the art as being encompassed by the scope of the presently disclosed and enabled invention. Also, it is to be understood that the phraseology and terminology employed herein is for the purpose of description and should not be regarded as limiting.

Angiostatin and “angiostatin-like” compounds such as other kringles of plasminogen (i.e. angiostatin precursors), as well as PEDF and SLED compounds, are potent angiogenic inhibitors. Such angiogenic inhibitory compositions are disclosed herein as also being particularly useful in inhibiting vascular leakage, inflammation and fibrosis. In one embodiment, angiostatin and PEDF have been found to have particular effects on retinal vascular leakage, inflammation and fibrosis, which are associated with diabetic macular edema, tumor growth and inflammation. The oxygen-induced retinopathy and streptozotocin-induced diabetic rat models showed significantly increased vascular permeability in the retina. A single dose intravitreal injection of angiostatin or PEDF reduced vascular permeability in the retina of rats with oxygen-induced retinopathy in a dose-dependent manner. This effect occurred at one and two days following the injection. No apparent inhibition of retinal neovascularization was observed at these early stages, suggesting that the reduced vascular permeability is not due to inhibition of retinal neovascularization. Angiostatin and PEDF also significantly reduced vascular leakage in the retina and iris of diabetic rats, but did not affect vascular permeability of normal rats. Western blot analysis and immunohistochemistry both showed that angiostatin and PEDF down-regulated retinal VEGF expression in both rat models but not in normal controls. The above-identified data reveal that angiostatin, PEDF and other like compounds have a significant therapeutic component to their activity, i.e., in reducing pathologic vascular leakage, inflammation and fibrosis, which is independent of its anti-angiogenic activity. This effect is mediated, at least in part, via blockage of VEGF over-expression under hypoxia.

In another embodiment of the present invention, angiostatin and angiostatin-like compounds, as well as PEDF and SLED compounds, have been shown herein to be useful in inhibiting vascular leakage, inflammation and fibrosis in diabetic retinopathy as well as in additional organs or systems, such as diabetic nephropathy, proteinuria from the kidney, brain edema, chronic inflammation, edema, arthritis, uveitis, macular degeneration, ascites, kidney inflammatory disease, disorders resulting in kidney fibrosis, hyperglycemia and the like. Expression of angiostatin and PEDF have been shown herein to be decreased at both the mRNA and protein levels in the kidneys of diabetic rats, while TGF-β and fibronectin levels were increased in the same diabetic kidneys. As shown by immunohistochemistry, the decreases in angiostatin expression and PEDF expression occur primarily in the glomeruli. In vitro studies have shown herein that high concentrations of glucose significantly decreased both angiostatin secretion and PEDF secretion in primary human mesangial cells (HMC), suggesting that hyperglycemia is a direct cause of angiostatin and PEDF decreases in the kidney. Toward the function of PEDF, it has been shown herein that either angiostatin or PEDF can block the high glucose-induced over-expression of TGF-β, a major pathogenic factor in diabetic nephropathy, and fibronectin in primary HMC, suggesting that angiostatin and PEDF may function as endogenous inhibitors of TGF-β expression and fibronectin production in glomeruli. Therefore, decreased expression of angiostatin and/or PEDF in diabetic kidney may contribute to extracellular matrix overproduction and the development of diabetic nephropathy.

Further, experiments herein demonstrate that PEDF reduction of albuminuria in diabetic rats may be obtained by administration of the protein directly or by administration of a DNA molecule encoding such protein, thereby providing multiple mechanisms of administration of the compositions utilized in the methods of the present invention.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)) and Coligan et al. Current Protocols in Immunology (Current Protocols, Wiley Interscience (1994)), which are expressly incorporated herein by reference in their entirety. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

The term “isolated protein” referred to herein means a protein of cDNA, recombinant RNA, or synthetic origin or some combination thereof, which by virtue of its origin, or source of derivation, the “isolated protein” (1) is not associated with proteins found in nature, (2) is free of other proteins from the same source, e.g. free of murine proteins, (3) is expressed by a cell from a different species, or (4) does not occur in nature.

The term “polypeptide” as used herein is a generic term to refer to native-protein, fragments, or analogs of a polypeptide sequence. Hence, native protein, fragments, and analogs are species of the polypeptide genus.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is referred to herein as “naturally-occurring”.

The term “isolated polynucleotide” as used herein shall mean a polynucleotide of genomic, cDNA, or synthetic origin or some combination thereof, which by virtue of its origin the “isolated polynucleotide” (1) is not associated with all or a portion of a polynucleotide in which the “isolated polynucleotide” is found in nature, (2) is operably linked to a polynucleotide which it is not linked to in nature, or (3) does not occur in nature as part of a larger sequence.

The term “polynucleotide” as referred to herein means a polymeric form of nucleotides of at least 10 bases in length, either ribonucleotides or deoxynucleotides or a modified form of either type of nucleotide. The term includes single and double stranded forms of DNA.

The term “naturally occurring nucleotides” referred to herein includes deoxyribonucleotides and ribonucleotides. The term “modified nucleotides” referred to herein includes nucleotides with modified or substituted sugar groups and the like. The term “oligonucleotide linkages” referred to herein includes oligonucleotides linkages such as phosphorothioate, phosphorodithioate, phosphoroselenoate, phosphorodiselenoate, phosphoroanilothioate, phoshoraniladate, phosphoroamidate, and the like. See e.g., LaPlanche et al. Nucl. Acids Res. 14:9081 (1986); Stec et al. J. Am. Chem. Soc. 106:6077 (1984); Stein et al. Nucl. Acids Res. 16:3209 (1988); Zon et al. Anti-Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990), the disclosures of which are hereby incorporated by reference. An oligonucleotide can include a label for detection, if desired.

The term “selectively hybridize” referred to herein means to detectably and specifically bind. Polynucleotides, oligonucleotides and fragments thereof in accordance with the invention selectively hybridize to nucleic acid strands under hybridization and wash conditions that minimize appreciable amounts of detectable binding to nonspecific nucleic acids. High stringency conditions can be used to achieve selective hybridization conditions as known in the art and discussed herein. Generally, the nucleic acid sequence homology between the polynucleotides, oligonucleotides, and fragments of the invention and a nucleic acid sequence of interest will be at least 80%, and more typically with preferably increasing homologies of at least 85%, 90%, 95%, 99%, and 100%. Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. See Dayhoff, M. O., in Atlas of Protein Sequence and Structure, pp. 101-110 (Volume 5, National Biomedical Research Foundation (1972)) and Supplement 2 to this volume, pp. 1-10. The two sequences or parts thereof are more preferably homologous if their amino acids are greater than or equal to 50% identical when optimally aligned using the ALIGN program. The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The following terms are used to describe the sequence relationships between two or more polynucleotide or amino acid sequences: “reference sequence”, “comparison window”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA or gene sequence given in a sequence listing or may comprise a complete cDNA or gene sequence. Generally, a reference sequence is at least 18 nucleotides or 6 amino acids in length, frequently at least 24 nucleotides or 8 amino acids in length, and often at least 48 nucleotides or 16 amino acids in length. Since two polynucleotides or amino acid sequences may each (1) comprise a sequence (i.e., a portion of the complete polynucleotide or amino acid sequence) that is similar between the two molecules, and (2) may further comprise a sequence that is divergent between the two polynucleotides or amino acid sequences, sequence comparisons between two (or more) molecules are typically performed by comparing sequences of the two molecules over a “comparison window” to identify and compare local regions of sequence similarity. A “comparison window”, as used herein, refers to a conceptual segment of at least 18 contiguous nucleotide positions or 6 amino acids wherein a polynucleotide sequence or amino acid sequence may be compared to a reference sequence of at least 18 contiguous nucleotides or 6 amino acid sequences and wherein the portion of the polynucleotide sequence in the comparison window may comprise additions, deletions, substitutions, and the like (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman Adv. Appl. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85:2444 (1988), by computerized implementations of these algorithms (GAP, BESTFIT, FASTA, and TFASTA in the Wisconsin Genetics Software Package Release 7.0, (Genetics Computer Group, 575 Science Dr., Madison, Wis.), Geneworks, or MacVector software packages), or by inspection, and the best alignment (i.e., resulting in the highest percentage of homology over the comparison window) generated by the various methods is selected.

The term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U, or I) or residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has at least 85 percent sequence identity, preferably at least 90 to 95 percent sequence identity, more usually at least 99 percent sequence identity as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence.

As used herein, the twenty conventional amino acids and their abbreviations follow conventional usage. See Immunology—A Synthesis (2.sup.nd Edition, E. S. Golub and D. R. Gren, Eds., Sinauer Associates, Sunderland, Mass. (1991)), which is incorporated herein by reference. Stereoisomers (e.g., D-amino acids) of the twenty conventional amino acids, unnatural amino acids such as .alpha.-, .alpha.-disubstituted amino acids, N-alkyl amino acids, lactic acid, and other unconventional amino acids may also be suitable components for polypeptides of the present invention. Examples of unconventional amino acids include: 4-hydroxyproline, .gamma.-carboxyglutamate, .epsilon.-N,N,N-trimethyllysine, .epsilon.-N-acetyllysine, O-phosphoserine, N-acetylserine, N-formylmethionine, 3-methyihistidine, 5-hydroxylysine, .sigma.-N-methylarginine, and other similar amino acids and imino acids (e.g., 4-hydroxyproline). In the polypeptide notation used herein, the lefthand direction is the amino terminal direction and the righthand direction is the carboxy-terminal direction, in accordance with standard usage and convention.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity, and most preferably at least 99 percent sequence identity. Preferably, residue positions which are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, glutamic-aspartic, and asparagine-glutamine.

As discussed herein, minor variations in the amino acid sequences of compositions having inhibition of vascular leakage, inflammation and fibrosis activities are contemplated as being encompassed by the present invention, providing that the variations in the amino acid sequence maintain at least 75%, more preferably at least 80%, 90%, 95%, and most preferably 99%. In particular, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Fragments or analogs of proteins or peptides of the present invention can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, the foregoing examples demonstrate that those of skill in the art can recognize sequence motifs and structural conformations that may be used to define structural and functional domains in accordance with the invention.

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various mutations of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et at. Nature 354:105 (1991), which are each incorporated herein by reference.

The term “polypeptide fragment” as used herein refers to a polypeptide that has an amino-terminal and/or carboxy-terminal deletion, but where the remaining amino acid sequence is identical to the corresponding positions in the naturally-occurring sequence deduced, for example, from a full-length cDNA sequence. Fragments typically are at least 5, 6, 8 or 10 amino acids long, preferably at least 14 amino acids long, more preferably at least 20 amino acids long, usually at least 50 amino acids long, and even more preferably at least 70 amino acids long.

The term “pharmaceutical agent or drug” as used herein refers to a chemical compound or composition capable of inducing a desired therapeutic effect when properly administered to a patient. Other chemistry terms herein are used according to conventional usage in the art, as exemplified by The McGraw-Hill Dictionary of Chemical Terms (Parker, S., Ed., McGraw-Hill, San Francisco (1985)), incorporated herein by reference).

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

“Treatment” refers to both therapeutic treatment and prophylactic or preventative measures. Those in need of treatment include those already with the disorder as well as those in which the disorder is to be prevented.

A “disorder” is any condition that would benefit from treatment with the compositions exhibiting inhibition of at least one of vascular leakage, inflammation and fibrosis activities utilized in accordance with the methods of the present invention. This includes chronic and acute disorders or diseases including those pathological conditions which predispose the mammal to the disorder in question.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia. More particular examples of such cancers include squamous cell cancer, small-cell lung cancer, non-small cell lung cancer, gastrointestinal cancer, pancreatic cancer, glioblastoma, cervical cancer, ovarian cancer, liver cancer, bladder cancer, hopatoma, breast cancer, colon cancer, colorectal cancer, endometrial carcinoma, salivary gland carcinoma, kidney cancer, renal cancer, prostate cancer, vulval cancer, thyroid cancer, hepatic carcinoma and various types of head and neck cancer.

“Mammal” for purposes of treatment refers to any animal classified as a mammal, including human, domestic and farm animals, nonhuman primates, and zoo, sports, or pet animals, such as dogs, horses, cats, cows, etc. The term “patient” refers to human and veterinary subjects.

The term “effective-amount” refers to an amount of a biologically active molecule or conjugate or derivative thereof sufficient to exhibit a detectable therapeutic effect without undue adverse side effects (such as toxicity, irritation and allergic response) commensurate with a reasonable benefit/risk ratio when used in the manner of the invention. The therapeutic effect may include, for example but not by way of limitation, inhibiting permeability of vessels and other vasculature. The effective amount for a subject will depend upon the type of subject, the subject's size and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

As used herein, the term “concurrent therapy” is used interchangeably with the terms “combination therapy” and “adjunct therapy”, and will be understood to mean that the patient in need of treatment is treated or given another drug for the disease in conjunction with the compositions of the present invention. This concurrent therapy can be sequential therapy where the patient is treated first with one drug and then the other, or the two drugs are given simultaneously.

The term “pharmaceutically acceptable” refers to compounds and compositions which are suitable for administration to humans and/or animals without undue adverse side effects such as toxicity, irritation and/or allergic response commensurate with a reasonable benefit/risk ratio.

By “biologically active” is meant the ability to modify the physiological system of an organism. A molecule can be biologically active through its own functionalities, or may be biologically active based on its ability to activate or inhibit molecules having their own biological activity.

The compounds of the present invention may be administered to a subject by any method known in the art, including but not limited to, oral, topical, transdermal, parenteral, subcutaneous, intranasal, intramuscular, intraperitoneal, intravitreal and intravenous routes, including both local and systemic applications. In addition, the compounds of the present invention may be designed to provide delayed or controlled release using formulation techniques which are well known in the art.

The present invention also includes a pharmaceutical composition comprising a therapeutically effective amount of at least one of the compositions described herein above in combination with a pharmaceutically acceptable carrier. As used herein, a “pharmaceutically acceptable carrier” is a pharmaceutically acceptable solvent, suspending agent or vehicle for delivering the compounds of the present invention to the human or animal. The carrier may be liquid or solid and is selected with the planned manner of administration in mind. Examples of pharmaceutically acceptable carriers that may be utilized in accordance with the present invention include, but are not limited to, PEG, liposomes, ethanol, DMSO, aqueous buffers, oils, and combinations thereof.

The present invention is related to methods of inhibiting at least one of vascular leakage, inflammation and fibrosis due to a disease or disorder, such as but not limited to diabetes, by administration of an effective amount of a compound that exhibits inhibition of at least one of vascular leakage, inflammation and fibrosis, wherein at a substantially higher amount, such compound also exhibits anti-angiogenesis activity.

One compound that may be utilized in accordance with the present invention is a protein named “angiostatin”, which has been previously defined by its ability to overcome the angiogenic activity of endogenous growth factors such as bFGF, in vitro. Angiostatin comprises a protein having a molecular weight of between approximately 38 kilodaltons and 45 kilodaltons, as determined by reducing polyacrylamide gel electrophoresis, and having an amino acid sequence substantially similar to that of a fragment of plasminogen. Examples of angiostatin proteins that may utilized in accordance with the present invention are known in the art and are described in detail in U.S. Pat. No. 5,639,725, issued to O'Reilly et al. on Jun. 17, 1997; U.S. Pat. No. 5,733,876, issued to O'Reilly et al. on Mar. 31, 1998; U.S. Pat. No. 5,776,704, issued to O'Reilly et al. on Jul. 7, 1998; U.S. Pat. No.5,792,845, issued to O'Reilly et al. on Aug. 11, 1998; and U.S. Pat. No. 5,885,795, issued to O'Reilly et al. on Mar. 23, 1999; the contents of each of which are hereby expressly incorporated herein by reference in their entirety.

Other compounds that may be utilized in accordance with the present invention are fragments of angiostatin which retain the ability to inhibit at least one of vascular leakage, inflammation and fibrosis. Fragments of angiostatin that retain the anti-angiogenic activity of angiostatin are known in the art and are described in detail in U.S. Pat. No. 5,837,682, issued to Folkman et al. on Nov. 17, 1998; U.S. Pat. No. 5,854,221, issued to Cao et al. on Dec. 29, 1998; U.S. Pat. No. 5,945,403, issued to Folkman et al. on Aug. 31, 1999; and U.S. Pat. No. 6,024,688, issued to Folkman et al. on Feb. 17, 2000; the contents of each of which are hereby expressly incorporated herein by reference in their entirety. It is easily within the skill of a person having ordinary skill in the art to utilize the methods and teachings of the above-cited references regarding identification of anti-angiogenic fragments of angiostatin and adapt such methods and teachings to identify fragments of angiostatin that retain the ability to inhibit at least one of vascular leakage, inflammation and fibrosis, and therefore such currently unidentified active fragments of angiostatin are also fully within the scope of the present invention.

Other compounds that may be utilized in accordance with the present invention include allelic variants of angiostatin as well as any insertion, deletion or substitution mutants of angiostatin that retain the ability to inhibit at least one of vascular leakage, inflammation and fibrosis. Methods of identifying such variants or mutants of angiostatin are within the skill of a person having ordinary skill in the art and are therefore also within the scope of the present invention.

In another embodiment of the present invention, the composition Pigment Epithelium-Derived Factor (PEDF) may be utilized in accordance with the methods of the present invention. PEDF is a protein of the serine protease inhibitor (serpin) supergene family, and PEDF is a potent autocrine and paracrine hormone which blocks endothelial cell proliferation (including vascular endothelial cells, which are necessary for neovascularization), and promotes cellular differentiation, and is neurotrophic and neuroprotective. Examples of PEDF proteins and isoforms thereof are known in the art and disclosed in U.S. Pat. No. 6,204,248, issued to Demopoulos et al. on Mar. 20, 2001, the contents of which-are hereby expressly incorporated herein by reference in its entirety. Other examples of PEDF proteins that may be utilized in accordance with the present invention are disclosed in U.S. Pat. No. 6,228,024, issued to Bouck et al. on Sep. 11, 2001; U.S. Pat. No. 6,391,850, issued to Bouck et al. on May 21, 2002; and U.S. Pat. No. 6,670,333, issued to Bouck et al. on Dec. 30, 2003; the contents of each of which are hereby expressly incorporated herein by reference in their entirety. The above-referenced patents disclose SLED proteins and peptides, which include any antiangiogenic derivative of PEDF, including but not limited to, full length PEDF, allelic variants of PEDF, any insertion, deletion or susbtitution mutants of PEDF, and derivatives thereof. Therefore, any PEDF or SLED compound disclosed herein or known in the art is fully within the scope of the methods of the present invention.

Other angiostatin-like compounds or PEDF-like compounds and other anti-angiogenic factors may be utilized in accordance with the present invention. Examples of such compounds include, but are not limited to, other fragments of plasminogen, as disclosed in U.S. Pat. No. 6,521,439, issued to Folkman et al. on Feb. 18, 2003; endostatin (a C terminal 20 kD fragment of the basement membrane protein Collagen XVIII), as disclosed in U.S. Pat. No. 5,854,205, issued to O'Reilly et al. on Dec. 29, 1998; U.S. Pat. No.6,346,510, issued to O'Reilly et al. on Feb. 12, 2002; U.S. Pat. No. 6,630,448, issued to O'Reilly et al. on Oct. 7, 2003; U.S. Pat. No. 6,746,865, issued to O'Reilly et al. on Jun. 8, 2004; and U.S. Pat. No. 6,764,995, issued to O'Reilly et al. on Jul. 20, 2004; antithrombin III, as disclosed in U.S. Pat. No. 6,607,724, issued to O'Reilly et al. on Aug. 19, 2003; other fumagillin derivatives, such as but not limited to TNP-470, as disclosed in U.S. Pat. No.6,740,678, issued to Moulton et al. on May 25, 2004; thrombospondin, as disclosed in U.S. Pat. No. 4,610,960, issued to Mosha on Sep. 9, 1986; U.S. Pat. No. 5,190,918, issued to Deutch et al. on Mar. 2, 1993; and U.S. Pat. No. 5,192,744, issued to Bouck et al. on Mar. 9, 1993; platelet factor 4, as disclosed in U.S. Pat. No. 5,304,542, issued to Tatakis on Apr. 19, 1994; U.S. Pat. No. 5,436,222, issued to Kun et al. on Jul. 25, 1995; and U.S. Pat. No. 5,512,550, issued to Gupta et al. on Apr. 30, 1996; maspin, as disclosed in U.S. Pat. No. 5,905,023, issued to Sager et al. on May 18, 1999; and U.S. Pat. No. 5,470,970, issued to Sager et al. on Nov. 28, 1995; tumostatin, and other like compounds. The contents of each of the references listed herein above are hereby expressly incorporated herein by reference in their entirety. Further, one of ordinary skill in the art will appreciate that any compound described herein can be modified or truncated and retain the desired inhibition of at least one of vascular leakage, inflammation and fibrosis activities. As such, active fragments of the compounds described herein are suitable for use in the present inventive methods.

Therefore, the term “angiostatin” or“PEDF” as used herein will be understood to refer to angiostatin or PEDF as described herein above, peptide fragments of angiostatin or PEDF that have at least one of vascular leakage-, inflammation- and fibrosis-inhibiting activities; and analogs or derivatives of angiostatin or PEDF that have substantial sequence homology (as defined herein) to the amino acid sequence of angiostatin or PEDF, respectively, which have at least one of vascular leakage-, inflammation- and fibrosis-inhibiting activities.

The angiostatin and PEDF proteins utilized in accordance with the present invention may be isolated from body fluids, such as but not limited to blood or urine. Optionally, the angiostatin or PEDF proteins utilized in accordance with the present invention may be synthesized by recombinant, enzymatic or chemical methods. Such recombinant, enzymatic and chemical methods are fully within the skill of a person of ordinary skill in the art, and thus angiostatin or PEDF proteins produced by such methods are fully within the scope of the present invention. When recombinant methods of producing angiostatin or PEDF are utilized in accordance with the present invention, the angiostatin or PEDF may be in a solubilized, refolded form, or the angiostatin or PEDF may be in the form of an aggregate. For example but not by way of limitation, when aggregate angiostatin is produced after purification and used directly, without further renaturing, reducing or alkylating, the product provides a means of sustained release of angiostatin, thereby optimizing its efficiency, as disclosed in U.S. Pat. No. 5,861,372, issued to Folkman et al. on Jan. 19, 1999, the contents of which are hereby expressly incorporated herein by reference in its entirety.

Preferred methods of administration of the compositions described herein above in accordance with the methods of the present invention include oral, topical, transdermal, parenteral, subcutaneous, intranasal, intramuscular, intraperitoneal, intravitreal and intravenous routes, including both local and systemic applications. In addition, the compositions of the present invention may be designed to provide delayed or controlled release using formulation techniques which are well known in the art.

The amount of the compositions of the present invention required to exhibit the inhibition of vascular leakage activity in an animal may be at least 10-fold lower than the amount required to exhibit the anti-angiogenic activity of the composition, and preferably may be at least 50-fold lower than the amount required to exhibit the anti-angiogenic activity of the composition, and more preferably may be at least 100-fold lower than the amount required to exhibit the anti-angiogenic activity of the composition.

Further, the methods of the present invention also envisage administration of an isolated nucleotide sequence, such as a DNA molecule, encoding a protein or peptide capable of inhibiting at least one of vascular leakage, inflammation and fibrosis, such as but not limited to, a DNA encoding angiostatin, PEDF, an angiostatin-like compound, a PEDF-like compound, a fragment or derivative of angiostatin or PEDF, or combinations thereof. Such DNA molecules are described in the references incorporated herein above, and it is within the skill of a person having ordinary skill in the art to identify and administer DNA molecules that could be utilized in accordance with the present invention.

It is to be understood that while certain protein and/or DNA compositions are described herein as being utilized with the methods of the present invention, the methods of the present invention are not limited to the use of such compositions. Based on the large amount of information available in the art, as evidenced herein above by the patents incorporated herein, as well as the general knowledge in the field of anti-angiogenic compounds, one of ordinary skill in the art could easily utilize other known anti-angiogenic compounds having the characteristics described herein in the methods of the present invention, and therefore the use of other similar anti-angiogenic compounds that are capable of inhibiting vascular leakage, inflammation and/or fibrosis also falls within the scope of the present invention.

The invention is further illustrated by the following examples, which are not to be construed in any way as imposing limitations upon the scope of the present invention. On the contrary, it is to be clearly understood that various other embodiments, modifications, and equivalents thereof, after reading the description herein in conjunction with the Drawings and appended claims, may suggest themselves to those skilled in the art without departing from the spirit and scope of the presently disclosed and claimed invention.

### EXAMPLE 1

### Effect of Angiostatin on Vascular Leakage and VEGF-Expression in Rat Retina

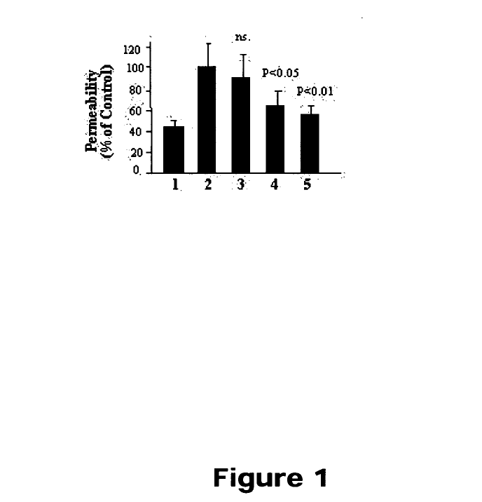

Now referring to the Figures, FIG. 1 illustrates that angiostatin reduces vascular permeability in the retina of OIR rats. Previous studies have shown that OIR rats have a transient increase of retinal vascular permeability with the peak at age P16 (Zhang et al., 2004). To determine the effect of angiostatin on vascular permeability, OIR rats (P14) received an intravitreal injection of 3 μl of angiostatin with different concentrations into, the right eyes, to reach doses of 1.875, 3.75 and 7.5 μg/eye. The same volume of PBS was injected into the left eyes for controls. Vascular permeability was measured at P16 using the Evans blue method. In the eyes injected with angiostatin, vascular permeability was reduced in an angiostatin dose-dependent manner. At doses of 3.75 and 7.5 μg/eye, angiostatin decreased the permeability to approximately 70% and 50%, respectively, of the contralateral control with PBS injection (P<0.05 and P<0.01, respectively, n=4), while the low dose of angiostatin (1.875 μg/eye) showed no significant reduction in permeability (P>0.05, n=4). No significant reduction of vascular permeability was detected in the iris of OIR rats treated with angiostatin at all the doses used.

To determine the effect of angiostatin on retinal neovascularization, angiostatin was injected into the vitreous of the right eye (7.5 μg/eye) of OIR rats at P12 and PBS into the left eye. Retinal neovascularization was examined at P18 using both the fluorescein angiography on retinal whole mounts and by quantifying pre-retinal vascular cells as described previously (Smith et al., 1994). Both results showed that angiostatin, at the dose used, had no detectable effect on retinal neovascularization at this early time point, suggesting that the angiostatin-induced reduction of vascular permeability is not a consequence of inhibition of retinal neovascularization.

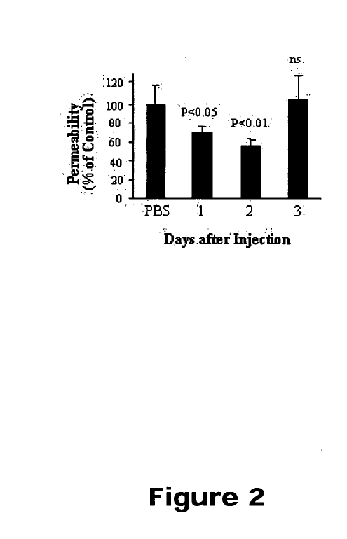

FIG. 2 illustrates the time course of angiostatin-induced reduction of vascular permeability in OIR rat retina. OIR rats received an intravitreal injection of 7.5 μg/eye of angiostatin at P14 in the right eye and PBS in the left eye. Vascular permeability was measured at P15, P16 and P17. The injection of angiostatin reduced retinal vascular permeability to approximately 70% and 50% of the contralateral control (P<0.05 and P<0.01, respectively, n=4) at P15 and P16, respectively. At P17, 3 days after the injection, vascular permeability returned to the level of the PBS-injected contralateral control. No significant reduction of vascular permeability was observed in the iris at these time points.

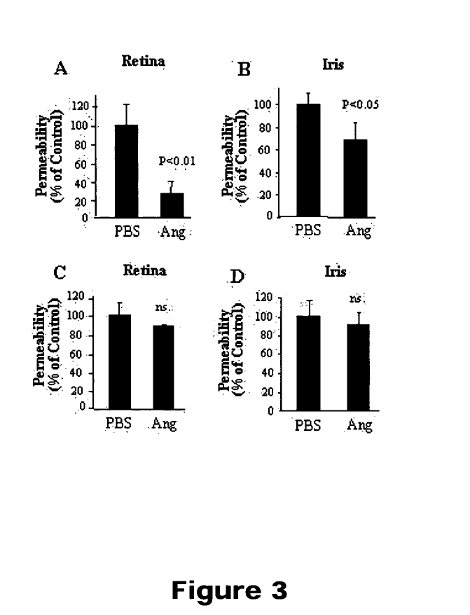

FIG. 3 illustrates the effect of angiostatin on retinal vascular leakage in diabetic rats. The STZ-diabetic rats received an intravitreal injection of angiostatin (7.5 μg/eye) in the right eye and PBS in the left eye at 2 weeks following the onset of diabetes. Vascular permeability in the retina and iris was measured 2 days after the injection. Angiostatin significantly decreased vascular permeability to 30% of the control with PBS injection (P<0.01, n=4) in the retina and to 70% of the control in the iris (P<0.05, n=4) (FIGS. 3A & 3B). In contrast, intravitreal injection of the same dose of angiostatin did not result in any significant reduction of vascular permeability in the retina and iris of normal rats, when compared with the contralateral eye with PBS injection (P>0.05, n=4) (FIGS. 3C&D).

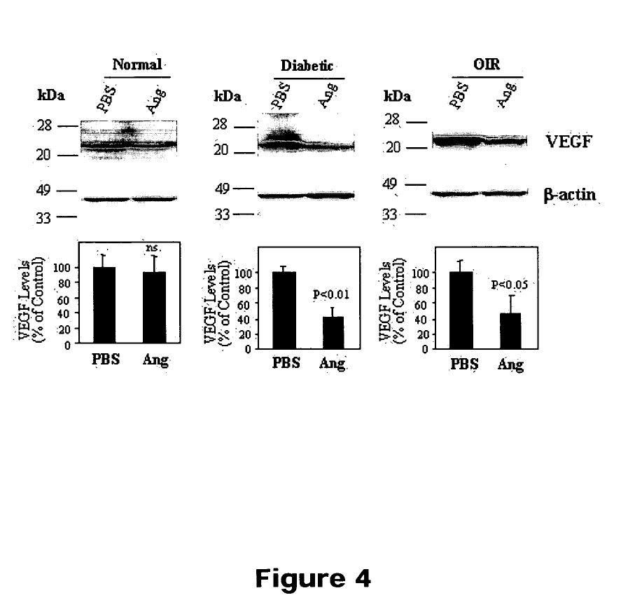

FIG. 4 illustrates that angiostatin down-regulates VEGF expression in the retina of the STZ-diabetic and OIR rats but not in normal rats. As over-expression of VEGF is known as a major cause of vascular hyper-permeability, the effect of angiostatin on VEGF expression was determined in OIR, STZ-diabetic and normal rats. Angiostatin was injected into the vitreous of the right eyes (7.5 μg/eye) and PBS into the left eyes of OIR rats at age P14, or into STZ-diabetic rats at 2 weeks after the onset of diabetes and age-matched normal adult rats. One day following the injection, VEGF levels in the retina were measured by Western blot analysis using an antibody specific for VEGF. Angiostatin decreased VEGF levels by approximately 2.5-fold and 2-fold in the retinas of the OIR and STZ-diabetic rats, respectively, but not in normal rats, correlating with its effect on vascular permeability.

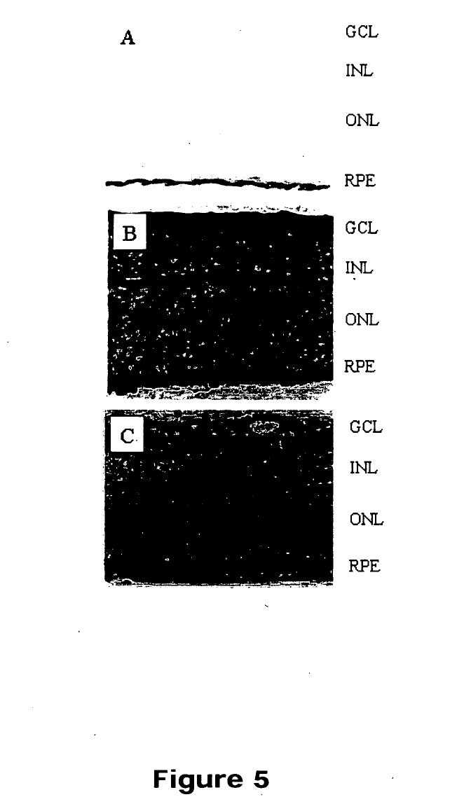

Immunohistochemistry using the anti-VEGF antibody demonstrated that angiostatin decreased the intensity of VEGF signals in the retina of the OIR rats, one day following the injection, when compared to the PBS-injected contralateral eye (FIG. 5).

Angiostatin has been shown to inhibit endothelial cells (O'Reilly et al., 1994). In the present invention, a new activity of angiostatin has been identified, i.e., reducing pathological vascular permeability in the retinas of both OIR and STZ-diabetic rat models but not in normal rats. Further, the results of Example 1 of the present invention for the first time showed that angiostatin down-regulates VEGF expression in the retinas of both the OIR and STZ-diabetic models, but not in normal rat retina, correlating with its effect on vascular permeability. These findings suggest that the angiostatin-induced reduction in vascular permeability may be ascribed, at least in part, to its down-regulation of VEGF expression.

Recently, it has been shown that the BRB is compromised in OIR rats (Zhang et al., 2004). Therefore, the effect of angiostatin on vascular permeability was tested using both OIR as well as STZ-diabetes models. It has been shown that angiostatin-induced inhibition of neovascularization in the OIR model occurs relatively late (P21) (Meneses et al., 2001). In contrast, the angiostatin-induced reduction of retinal vascular permeability in the same model can be detected as early as one day after the injection (FIG. 2). Analysis of retinal vasculature showed that angiostatin injection (7.5 μg/eye) did not result in any significant decrease of retinal neovascularization in the OIR model. To further confirm these findings, the effect of angiostatin on vascular permeability was also determined in STZ-induced diabetic rats which lack retinal neovascularization while showing a significant increase in vascular permeability (Antonetti et al., 1998). Angiostatin also significantly reduced vascular permeability in this STZ-diabetic animal model. Taken together, these results demonstrate that angiostatin-induced reduction in vascular permeability is not through its inhibition of neovascularization.

Although retinal edema in diabetes is a complex disorder, several lines of evidence suggest that VEGF plays a key role in vascular leakage in diabetic retina (Adamis et al., 1994; Aiello et al., 1994; and Pe'er et al., 1995). In OIR and STZ-diabetes models, retinal vascular leakage may be due to different structural changes in retinal capillaries. However, both models have increased VEGF levels in the retina, which is believed to play a key role in the development of vascular abnormalities in the retina (Hammes et al., 1998; and Pierce et al., 1995). VEGF is also known as a vasopermeability factor (Pierce et al., 1995; Battegay, 1995; and Dvorak et al., 1995) and is 50,000 times more potent than histamine in increasing dermal microvascular permeability (Senger et al., 1990). Over-expression of VEGF is associated with vascular leakage in diabetes (Aiello et al., 2000). Angiostatin blocks the over-expression of VEGF in the hypoxic retina as found in OIR and STZ-diabetes models but does not decrease the VEGF level in the normal retina (FIG. 4). Correlating with this observation, angiostatin only reduces retinal vascular permeability in OIR and STZ-diabetic rats but not in normal rats.

Other evidence demonstrating that angiostatin-induced reduction in vascular leakage is via blockade of VEGF production is that angiostatin did not reduce vascular hyper-permeability induced by an intravitreal injection of exogenous VEGF (data not shown). Taken together, these results demonstrate that the blockade of VEGF expression in hypoxic retina is responsible, at least in part, for the angiostatin-induced reduction of vascular leakage in OIR and STZ-diabetic rats.

It is unclear how angiostatin blocks VEGF expression at the present time. However, it has been shown that angiostatin binds to integrins (Tarui et al., 2001) and inhibits the activation of the p42/p44 MAP kinase pathway (Redlitz et al., 1999). As evidence has shown that the p42/p44 MAP kinase pathway plays a role in the regulation of VEGF expression and in angiogenesis control (Pages et al., 2000; and Milanini et al., 1998), the angiostatin-induced blockade of VEGF expression may be through inhibition of the MAP kinase pathway under hypoxia.

Breakdown of the BRB and/or vascular leakage is a major cause of macular edema in diabetic retinopathy and other ocular diseases such as uveitis (Ciulla et al., 1998; Bresnick, 1986; and Lopes de Faria et al., 1999). Current therapies for diabetic macular edema are not satisfactory, and macular edema is still a major cause of vision loss in diabetic patients. The present invention demonstrates that angiostatin can reduce vascular leakage in both diabetic and OIR rat models. Angiostatin down-regulates VEGF expression and thus, blocks the major cause of vascular leakage in diabetic retinas. Therefore, the angiostatin-induced reduction of vascular leakage may have therapeutic potential in the treatment of diabetic macular edema, cystoid macular edema and other diseases with vascular leakage such as uvietis and the wet form of macular degeneration.

### EXAMPLE 2

### Effect of Angiostatin on Vascular Leakage, TGF-β Expression and VEGF Expression in Diabetic Nephropathy

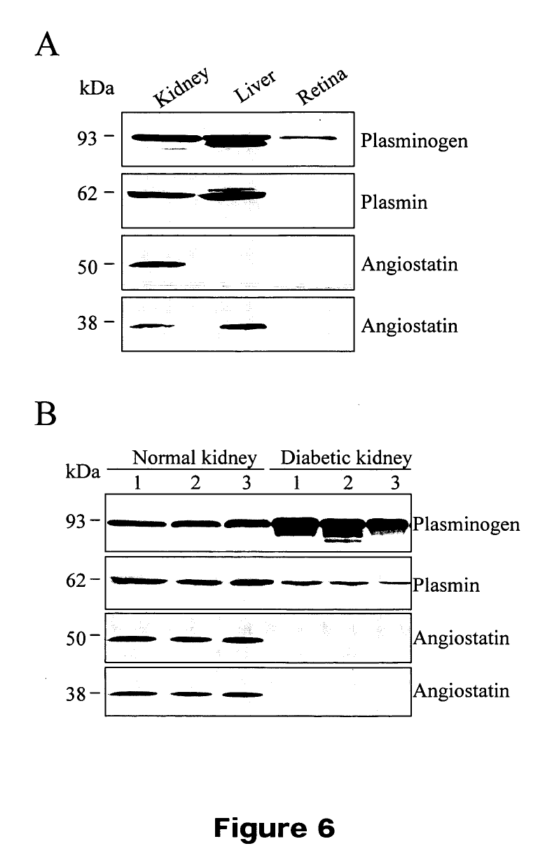

FIG. 6 illustrates the natural existence of angiostatin in rat kidney and a decrease in angiostatin levels in diabetic kidney. Angiostatin as an endogenous angiogenic inhibitor has been found at high levels in the serum and urine of cancer patients and tumor-bearing animals (O'Reilly et al., 1994; Cao, 1999). However, there are few reports on the levels of angiostatin in normal tissue, such as kidney, liver and retina. In Example 2 of the present invention, the existence of angiostatin was first demonstrated in the kidney as well as in the liver and retina in normal adult BN rats by Western blot. The results showed that high levels of plasminogen and proteolytic fragments existed in the kidney and liver (FIG. 6A). Two fragments of angiostatin of the molecular weight of 50 kDa and 38 kDa were found in the kidney, but only the 38 kDa angiostatin was found in the liver (FIG. 6A). In the retina, only low amounts of plasminogen but no proteolytic fragments thereof were detected at the concentrations assayed (FIG. 6A).

In the kidney of 6-week diabetic BN rats, the amount of angiostatin was significantly decreased when compared with that in the kidney of age-matched controls (FIG. 6B). The results also showed that in the same samples, plasminogen levels were much higher in the diabetic kidney than that in normal controls, indicating that the proteolysis of plasminogen and consequent production of plasminogen fragments including angiostatin were dramatically suppressed in diabetic kidney (FIG. 6B).

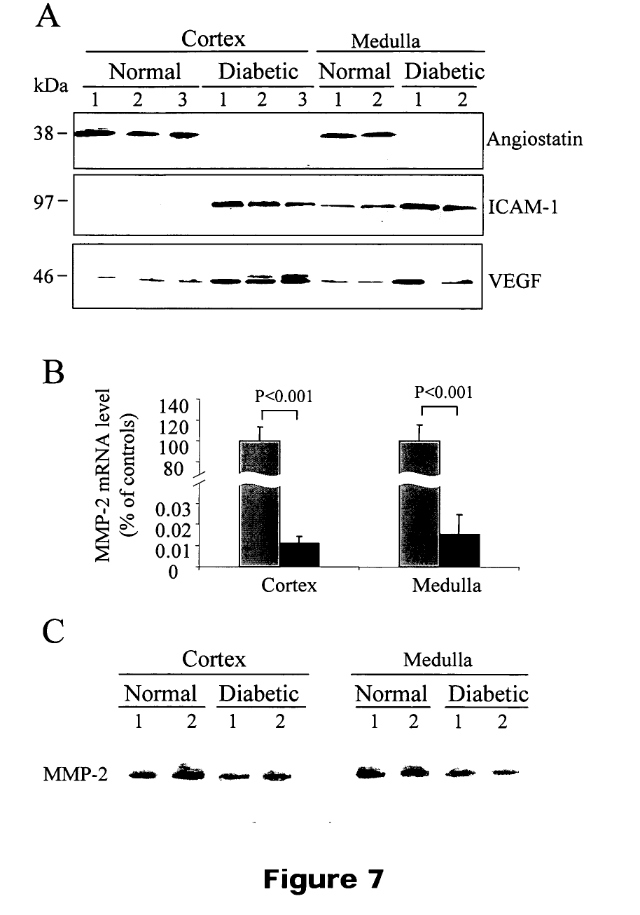

FIG. 7 illustrates decreased angiostatin levels and decreased MMP-2 expression in the diabetic cortex and medulla. To determine whether the decrease of angiostatin levels in diabetic kidney occurs in the cortex or in the medulla, or both, the cortex and medulla were carefully dissected from diabetic rats and age-matched normal controls. Angiostatin levels as well as ICAM-1 and VEGF levels were evaluated by Western blot. The results showed that angiostatin levels were significantly decreased in both diabetic cortex and diabetic medulla when compared with that in normal controls (FIG. 7A). In the same samples, ICAM-1 and VEGF levels showed dramatic increase in the diabetic tissues compared to the normal tissues, demonstrating that the decrease of angiogenic inhibitors and the increase of angiogenic and proinflammatory factors may be involved in the pathogenesis of DN.

To further explore the possible mechanism responsible for the decrease of angiostatin in diabetic kidney, the expression of matrix metalloproteinase (MMP-2), which was recognized as a major mediator for angiostatin production, was investigated in the kidneys of 6-week diabetic rats and normal controls. The results from real time RT-PCR demonstrated that mRNA levels of MMP-2 in diabetic cortex as well as in diabetic medulla were dramatically decreased when compared with that in the kidney of age-matched normal controls (FIG. 7B). Gelatin zymography showed that the gelatinolytic activity of MMP-2 was also significantly decreased in diabetic cortex and medulla when compared to that in normal controls (FIG. 7C). These results demonstrate that the decreased expression of MMP-2, which was believed to be a contributor to matrix protein accumulation and expansion in diabetic kidney, might also be at least partially responsible for the decrease of angiostatin levels in diabetic cortex and medulla.

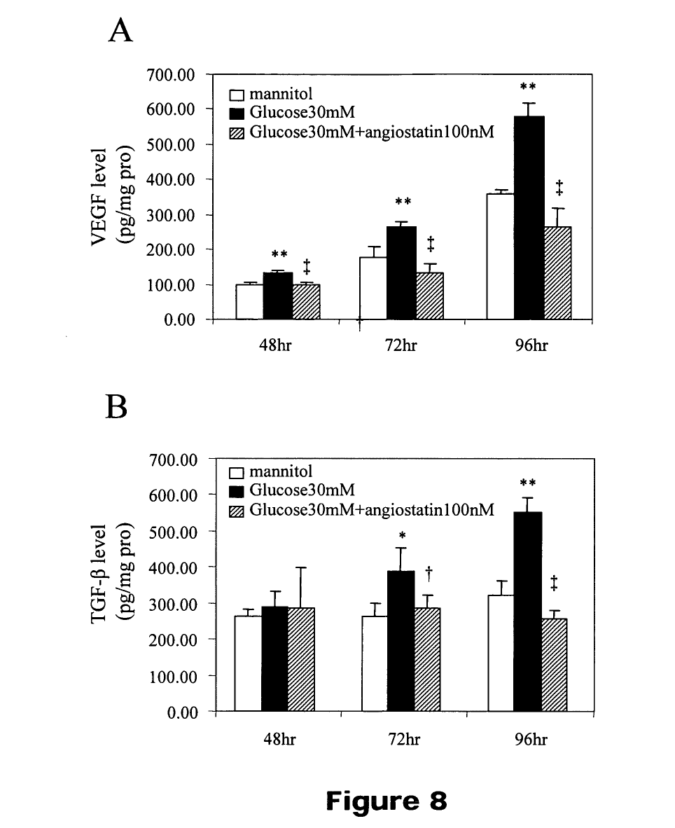

FIG. 8 demonstrates that angiostatin decreased the high glucose-induced increase in VEGF and TGF-β levels in cultured human mesangial cells. VEGF is a potent angiogenic and permeability factor, which is believed to be an important contributor to diabetic microvascular diseases, including DR and DN (Flyvberg, 2000; Flyvberg et al., 2002; and Bortoloso et al., 2001). It has been previously reported herein in Example 1 that angiostatin decreased VEGF levels in the retina in rats with STZ-induced diabetes (see FIGS. 4 and 5), and in Example 2 the effects of angiostatin on VEGF secretion in cultured human mesangial cells was determined. After incubation with high glucose (30 mM) for 48 h, VEGF was significantly increased, when compared with mannitol controls (FIG. 8A). Angiostatin at a concentration of 100 nM significantly decreased VEGF secretion to the control level. This effect was also observed at 72 h and 96 h after incubation (FIG. 8A).

As TGF-β is a well-known major mediator for the proliferation of mesangial cells and the overproduction of ECM in DN (Goldfarb et al., 2001; Tamaki et al., 2003; and Iglesias-de la Cruz et al., 2002), the effects of angiostatin on TGF-β secretion by HMC were also examined. After 48 h incubation with high glucose in the absence or presence of angiostatin, TGF-β levels in the cultured medium were comparable to that in the cells exposed to mannitol for osmolarity controls (FIG. 8B). However, after 72 h and 96 h incubation, TGF-β secretion was significantly increased in the cells treated with high glucose (FIG. 8B). Angiostatin at a concentration of 100 nM significantly inhibited high glucose-induced TGF-β secretion increase (FIG. 8B). This indicates that angiostatin inhibits glomerular fibrosis in DN.

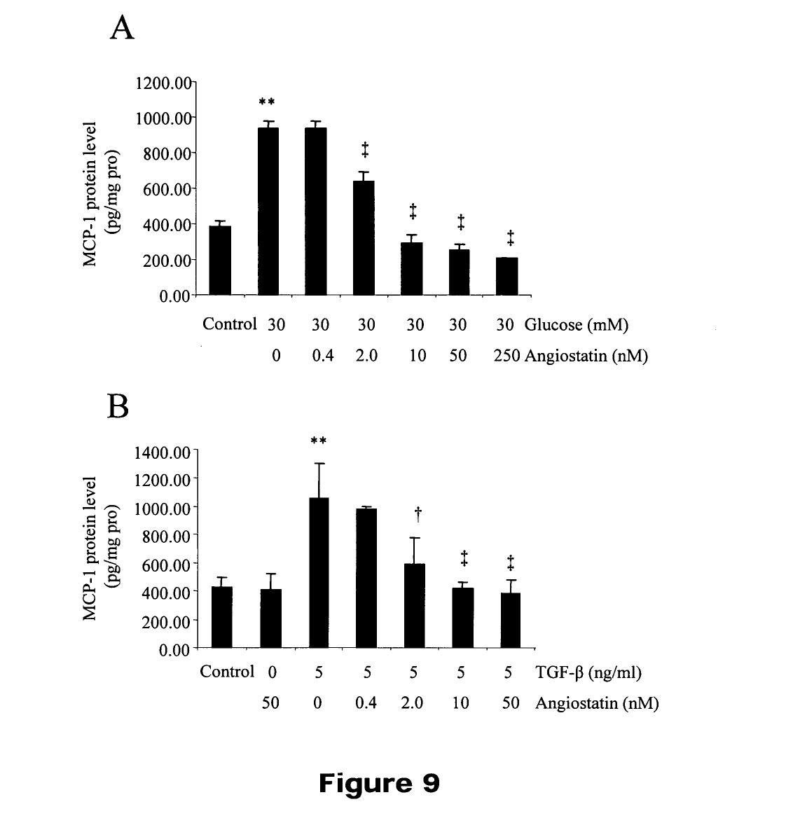

FIG. 9 illustrates that angiostatin decreased high glucose and TGF-β-induced MCP-1 secretion in cultured human mesangial cells. MCP-1 is one of the most important chemokines and inflammatory factors implicated in the pathogenesis of DN (Janssen et al., 2002; Wada et al., 2003; Amann et al., 2003; and Nishioka et al., 2001). High glucose activates NF-kappa B and further up-regulates MCP-1 expression in cultured mesangial cells (Ha et al., 2002). In the present invention, the effects of angiostatin on MCP-1 secretion induced by high glucose and TGF-β were examined. At 48 h after incubation with high glucose, the MCP-1 secretion increased by 2.5-fold when compared to the control (FIG. 9A). Angiostatin at doses from 2.0. nM to 250 nM significantly decreased MCP-1 secretion in a dose-dependent manner. At 48 h after TGF-β 5 ng/ml treatment, MCP-1 secretion was significantly increased (FIG. 9B). Angiostatin at doses from 2.0 nM to 50 nM significantly inhibited the increase in TGF-β-induced MCP-1 secretion in a dose-dependent manner. The highest dose of angiostatin had no effect on MCP-1 secretion in control cells (FIG. 9B), demonstrating that angiostatin only blocked the up-regulation of MCP-1 induced by TGF-β, but did not affect MCP-1 secretion under normal conditions.

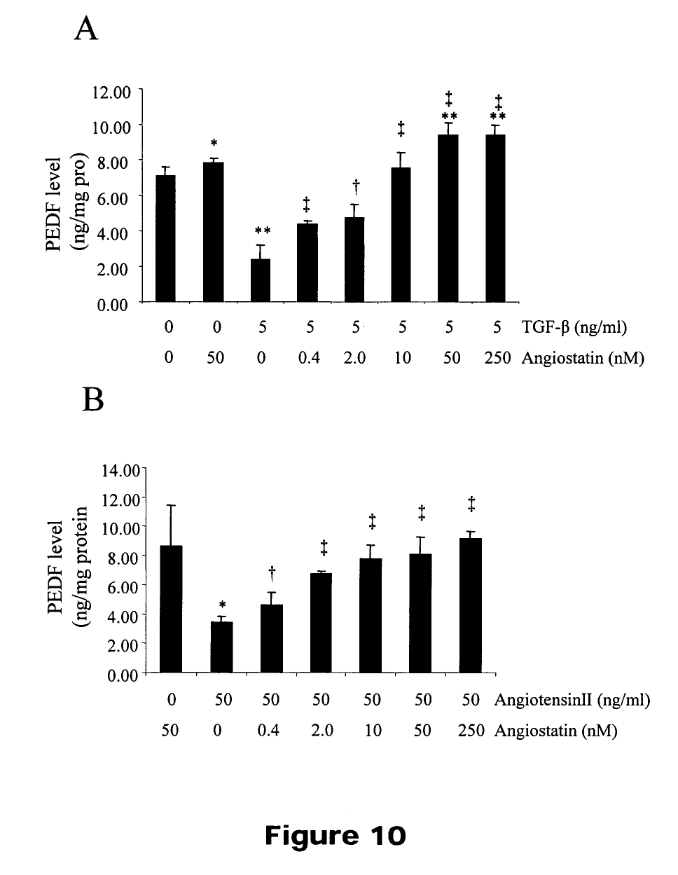

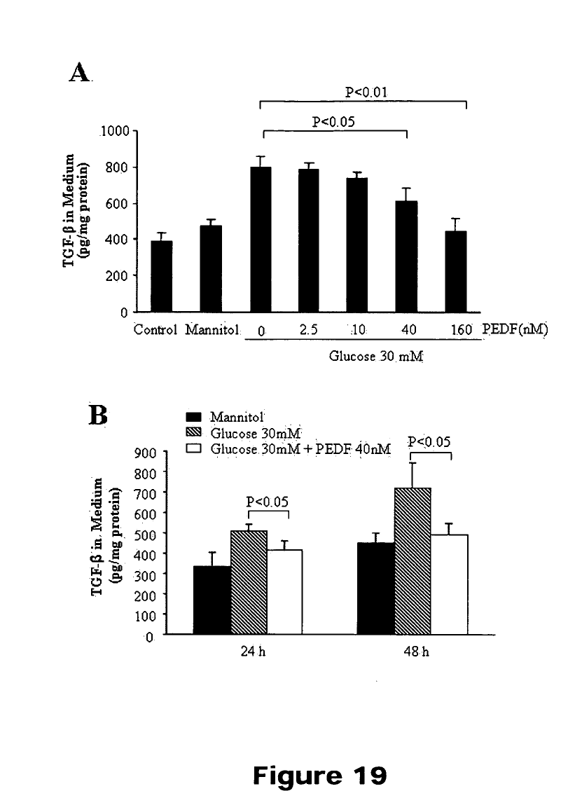

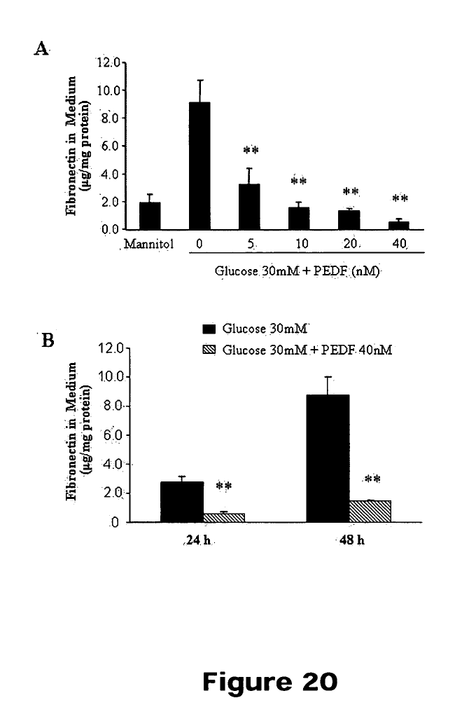

FIG. 10 illustrates that angiostatin inhibited high glucose-induced down-regulation of PEDF in cultured human mesangial cells. PEDF is a potent angiogenic inhibitor and neurotrophic factor, which has been widely studied in the eye as well as in other organs (Tombran-Tink et al., 1991; Dawson et al., 1999; and Tombran-Tink et al., 2003a). PEDF is shown herein after to inhibit high glucose-induced up-regulation of TGF-β and fibronectin production in cultured mesangial cells (see Example 3 and FIGS. 19 and 20). In the present example, the effect of angiostatin on PEDF secretion in cultured mesangial cells insulted by two commonly recognized pathological factors in diabetic renal diseases, TGF-β and angiotensin II (Goldfarb et al., 2001; Leehey et al., 2000), was evaluated.

After incubation with 5 ng/ml TGF-β for 48 h, PEDF secretion was significantly decreased by 3.5-fold when compared with control cells (FIG. 10A). Angiostatin at doses of 0.4 nM to 250 nM significantly inhibited the decrease of PEDF secretion induced by TGF-β in a dose-dependent manner (FIG. 10A). It was observed that PEDF levels in high dose angiostatin-treated cells were even higher than that in the control cells. Moreover, under normal conditions 50 nM angiostatin significantly increased PEDF secretion, suggesting angiostatin may be a potential regulation of PEDF secretion (FIG. 10A).

Angiotensin II has been shown to be a crucial factor in progressive glomerulosclerosis in DN through direct effects on glomerular cells by stimulating matrix protein synthesis and inhibiting degradation independent of its hemodynamic actions (Leehey et al., 2000). The results disclosed herein demonstrate that angiotensin II inhibited PEDF secretion in mesangial cells at doses from 12.5 ng/ml to 100 ng/ml. Angiostatin at concentrations of 2-250 nM effectively blocked the effects of angiotensin II on PEDF secretion (FIG. 10B).

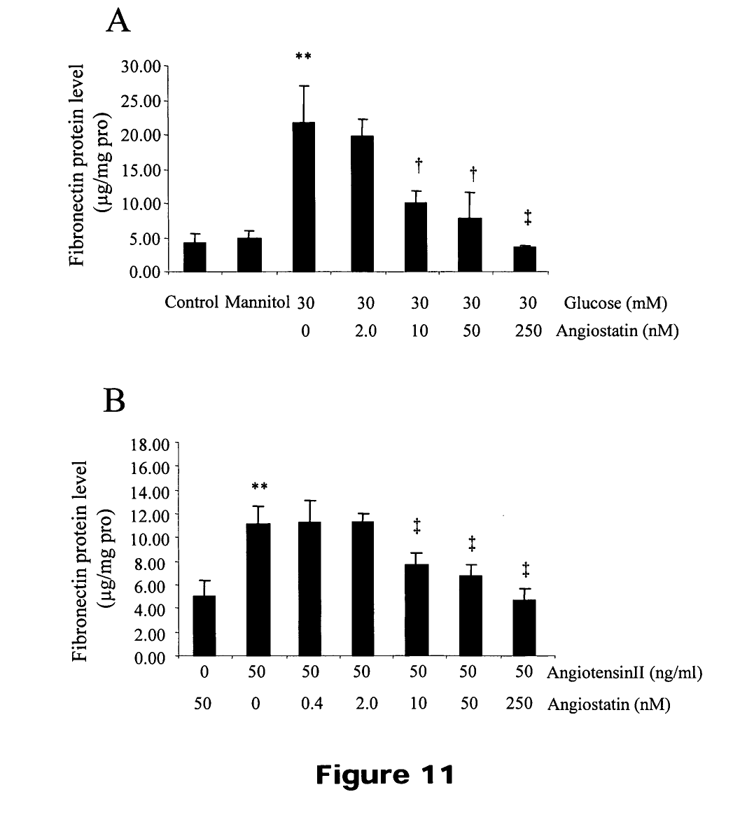

FIG. 11 illustrates that angiostatin inhibited high glucose and angiotensin II-induced fibronectin production in cultured mesangial cells. In the early stages of DN, overproduction of ECM proteins, such as fibronectin and collagen, is a major causative factor responsible for glomerular hyper-filtration and glomerular fibrosis (Weston et al., 2003). In cultured primary HMC, exposure to high glucose (30 mM) for 48 h led to significant increases in fibronectin secretion, compared to a mannitol control (FIG. 11A). At low doses (2-250 nM), angiostatin decreased the fibronectin secretion in a dose-dependent manner in HMC cultured in the high glucose medium (FIG. 11A). In cells exposed to 50 ng/ml of angiotensin II for 48 h, fibronectin production was dramatically increased (FIG. 11B). Angiostatin at doses of 10 nM to 250 nM significantly inhibited fibronectin secretion in a dose-dependent manner (FIG. 11B).

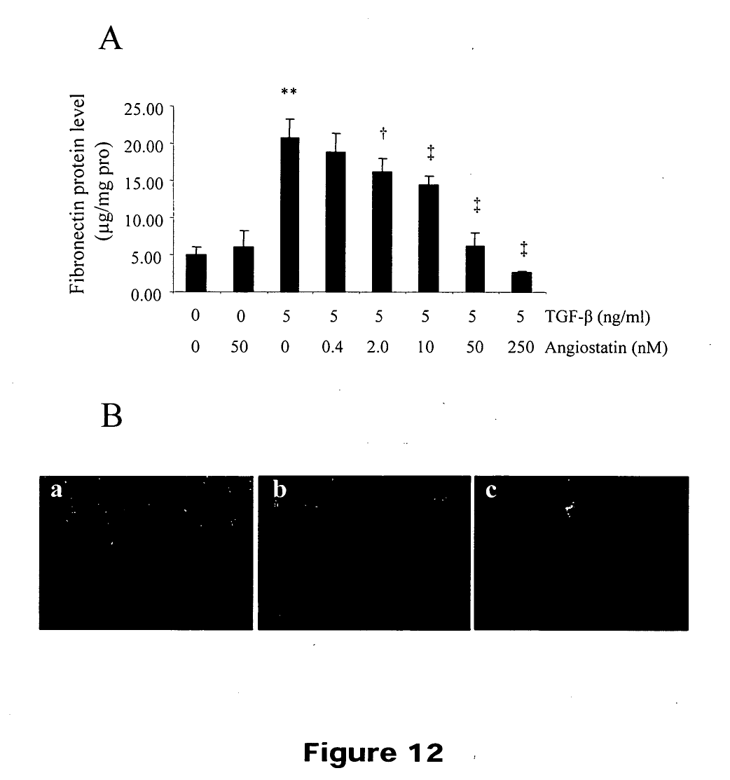

FIG. 12 illustrates that angiostatin suppressed TGF-β-induced fibronectin production by blocking Smad-2/3 nucleartranslocation in mesangial cells. As a crucial mediator in ECM production and accumulation, TGF-β strongly stimulated cultured mesangial cells to produce fibronectin. After incubation with TGF-β for 48 h, fibronectin secretion produced in HMC was increased by 4-fold when compared with controls. In the presence of different doses of angiostatin (0.4-250 nM), the effect of TGF-β was significantly abolished in a dose-dependent manner (FIG. 12A).

To further explore the possible mechanism underlying the effect of angiostatin on TGF-β-induced fibronectin production, a Smad-2/3 nuclear translocation assay was performed to elucidate whether the effect of angiostatin was caused by blockade of Smad activation. Cultured primary HMC were incubated with 5 ng/ml TGF-β in the absence or presence of 100 nM angiostatin for 1 h followed by an immunocytochemistry assay with anti-Smad-2/3 antibody. The results showed that TGF-β stimulated Smad-2/3 expression and translocation from the cytoplasm to the nuclei (FIG. 12B-b). The presence of 100 nM angiostatin significantly blocked the activation of Smad-2/3 induced by TGF-β (FIG. 12B-c).

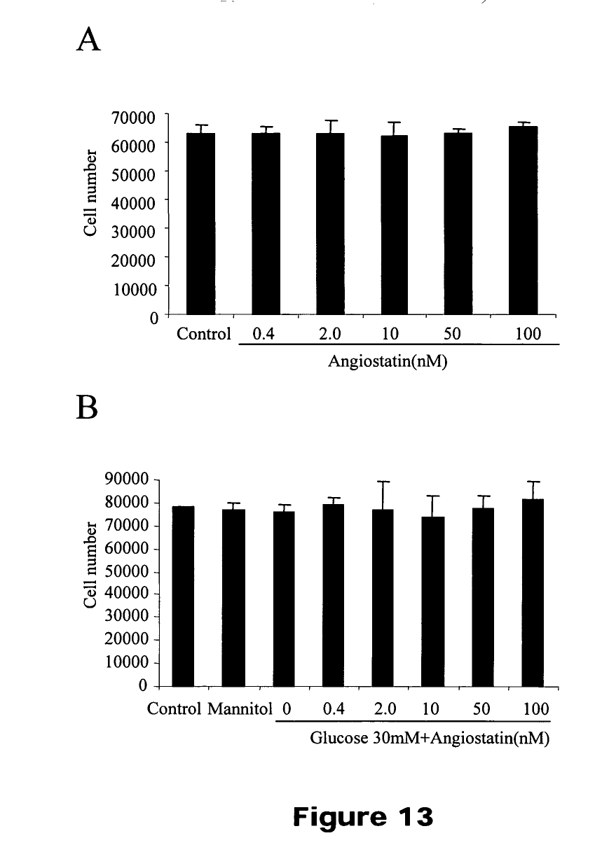

FIG. 13 illustrates that angiostatin does not affect the growth of HMC. To explore whether the effect of angiostatin on inhibition of fibronectin production is caused by an effect on the proliferation of mesangial cells, a cell proliferation assay was performed using the MTT method. The results showed that angiostatin did not affect the proliferation of cultured human mesangial cells under either high glucose conditions or normal glucose conditions (FIG. 13), thereby demonstrating that the effect of angiostatin on down-regulation of fibronectin and TGF-β levels is not through the inhibition of mesangial cell proliferation.

This Example demonstrates for the first time that endogenous angiostatin levels are significantly decreased in the kidney of the diabetic rat model. This decrease may be partially mediated by down-regulation of MMP-2 levels in the diabetic kidney. Further, the results demonstrate that angiostatin inhibited high glucose-induced increases in VEGF and TGF-β levels and also suppressed high glucose, angiotensin II and TGF-β-induced MCP-1 and fibronectin production in cultured human mesangial cells. These findings demonstrate that the decrease of angiostatin levels in the kidney may contribute to inflammation, fibrosis, proteinuria and renal injury in diabetes, and therefore angiostatin has great potential as a therapeutic agent in the prevention and treatment of DN.

Angiostatin was first identified as internal fragments of plasminogen in serum and urine of tumor-bearing animals (O'Reilly et al., 1994). Although angiostatin was given a single name, and subsequent studies have referred to angiostatin as a single species, in fact angiostatin contains several fragments of plasminogen with different biological activities (Cao et al., 2004). In the tissue extract for normal BN rats, two forms of angiostatin were observed at the molecular weight of 50 kDa and 38 kDa, consistent with the results reported by Basile and colleagues recently (Basile et al., 2004). Only one form of angiostatin at 38 kDa was observed in the liver. No angiostatin or other proteolytic fragments of plasminogen was observed in the retina at the concentrations assayed. When the same amount of total protein was blotted, plasminogen levels in the liver and kidney were much higher than that in the retina. The low abundance of plasminogen in the retina may be responsible for the non-detection of proteolytic fragments.

In the present Example 2, it has been determined whether angiostatin levels were changed in the kidney of 6-week diabetic rats, which had already developed the symptoms of nephropathy, including polyuria and microalbuminuria. The results showed that in diabetic rats, the proteolysis of plasminogen in the kidney was dramatically suppressed. The renal angiostatin levels as well as plasmin levels were significantly decreased, and the plasminogen level was significantly increased in diabetic kidney when compared to the age-matched normal controls. The angiostatin levels in the cortex and medulla of diabetic rats as well as control animals was further determined herein, and the results showed that angiostatin levels were decreased in the cortex and medulla of diabetic rats when compared with that in the normal controls, suggesting that the proteolysis of plasminogen was suppressed in diabetic cortex as well as medulla. Moreover, the levels of inflammatory factor ICAM-1 and the angiogenic factor VEGF in the diabetic cortex and medulla were significantly increased, indicating that the unbalance of angiogenic and inflammatory stimulators and inhibitors may be implicated in the pathogenesis of DN. Further, the possible mechanism responsible for the decrease of renal angiostatin levels in diabetic rats was explored. The expression of MMP-2, which has been shown to cleave plasminogen to release angiostatin, was dramatically down-regulated in diabetic cortex as well as medulla. The decreased activity of MMP-2 was also confirmed by gelatin zymography. These results demonstrate that the decrease of angiostatin levels in diabetic kidney might partially result from the decrease of MMP-2 expression.

Although angiostatin has been widely studied in a variety of tumor cells, the function of angiostatin in the kidney is largely unknown (Cao, 1999; O'Reilly et al., 1996; Cao et al., 2004; and Sim et al., 2000). To further elucidate the possible role of angiostatin in the pathogenesis of DN, cultured human mesangial cells were used as an in vitro model to investigate the effects of angiostatin on the major pathological factors in diabetic kidney. As VEGF and TGF-β are known to be up-regulated in the early stage of diabetic kidney and play a crucial role in formation of the pathological changes such as proliferation of mesangial cell and ECM production (Flyvbjerg, 2000; Khamaisi et al., 2003; and Zheng et al., 2002), the effects of angiostatin on VEGF and TGF-β expression in cultured mesangial cells were first tested. The results showed that in the cells treated with high glucose (30 mM), secretion of VEGF and secretion of TGF-β were significantly increased. Angiostatin at a concentration of 100 nM effectively inhibited high glucose-induced increases in VEGF and TGF-β levels, demonstrating that angiostatin affects the regulation of VEGF and TGF-β in the kidney. The decrease in angiostatin levels in diabetic kidney leads to the increase of VEGF and TGF-β levels, contributing to the pathological changes of kidney.

Pigment epithelium-derived factor (PEDF) is recognized as an anti-angiogenic factor and neurotrophic factor (Tombran-Tink et al., 2003a). The effects of angiostatin on PEDF expression in mesangial cells insulted by TGF-β or angiotensin II were determined herein. The results showed that both TGF-β and angiotensin II significantly decreased PEDF secretion in cultured mesangial cells. Angiostatin at low doses (2-250 nM) effectively inhibited high glucose and TGF-β-induced decrease in PEDF levels in a dose-dependent manner. More interestingly, under normal conditions angiostatin also increased PEDF secretion without interfering with cell proliferation. The mechanism underlying the effects of angiostatin on the up-regulation of PEDF is to be further elucidated.

Inflammation has been proposed as an important mediator in pathogenesis of DN (Kato et al., 1999; Janssen et al., 2002; Shestakova et al., 2002; and Okada et al., 2003). In the early stage of DN, several chemokines including MCP-1, TNF-α, ICAM-1, and IL-6 have been found to be up-regulated (Guler et al., 2002; Moriwaki et al., 2003; and Siragy et al., 2003). MCP-1 is a major chemokine produced by tubular epithelial cells and glomerular mesangial cells (Janssen et al., 2002; Wada et al., 2003; Amann et al., 2003; and Nishioka et al., 2001). MCP-1 induces monocyte immigration and differentiation to macrophages, which augment ECM production and tubular interstitial fibrosis in diabetic kidney (Amann et al., 2003). In the present Example, it has been demonstrated that angiostatin significantly blocked high glucose and TGF-α-induced MCP-1 secretion in mesangial cells in a dose-dependent manner, demonstrating that angiostatin can reduce inflammation in the diabetic kidney. These results were consistent with the previous studies on the anti-inflammatory effect of angiostatin (Benelli et al., 2003).

Over-production of ECM and mesangial matrix expansion is the character of pathological changes contributing to microalbuminuria in the early stage of DN and fibrosis (glomerularsclerosis) at late stage (Claesson-Welsh et al., 1998; and Makino et al., 1999). As mesangial cells are the major producers of ECM, primary HMC were used as a model to determine if angiostatin could block ECM protein fibronectin secretion induced by different stimulators, including high glucose, angiotensin II, and TGF-β. The results showed that high glucose at 30 mM, angiotensin II at 50 ng/ml, and TGF-β at 5 ng/ml significantly increased fibronectin secretion. Angiostatin blocked fibronectin over-production induced by different pathogens in a dose-dependent manner. Then it was determined whether the effect of angiostatin on fibronectin production was through the inhibition of mesangial cell proliferation. The results showed that angiostatin had no effect on mesangial cell growth rate. As Smad activation is the major signaling pathway mediating the function of TGF-β, it was further explored whether angiostatin blocked TGF-β function through inhibition of Smad activation. The results showed that angiostatin significantly blocked Smad-2/3 nuclear translocation, demonstrating that the effects of angiostatin on inhibition of TGF-β-induced inflammation and ECM production are at least partially through the blockade of Smad activation.

In summary, Example 2 of the present invention for the first time demonstrated that angiostatin is implicated in DN. Angiostatin plays an important role in prevention of mesangial ECM overproduction and pathological growth factor up-regulation in the kidney. The decreased angiostatin levels are involved in the pathogenesis of DN. Therefore, angiostatin has great therapeutic potential in the treatment of DN.

### EXAMPLE 3

### Effect of PEDF on Vascular Leakage, Vascular Permeability and Inflammation in Diabetic Nephropathy and Diabetic Retinopathy

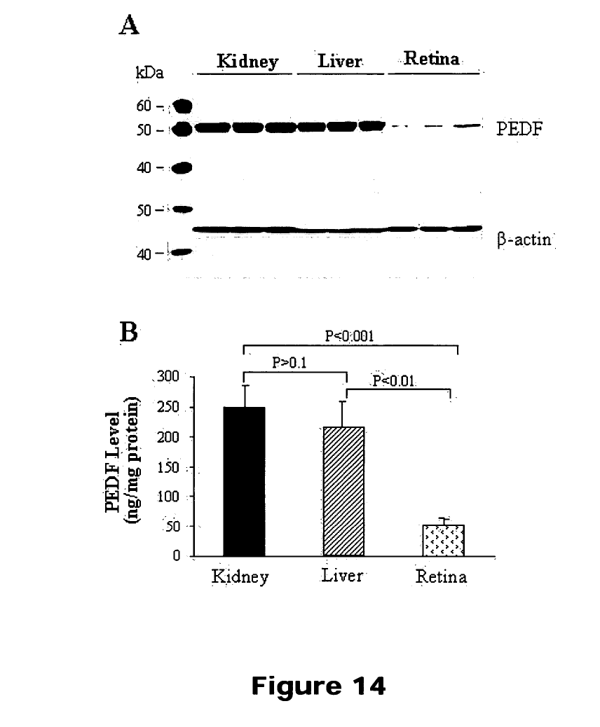

FIG. 14 illustrates high-level expression of PEDF in the kidney of normal rats. PEDF was recently found to be expressed in the kidney as well as in other organs, but its expression levels and cellular localization in the kidney have not been determined previously (Abramson et al., 2003). As the liver is regarded as the major source of systemic PEDF (Uehara et al., 2004; and Tombran-Tink et al., 1996), and the retina is a well-known site of PEDF expression and function, PEDF levels in the kidney were first compared with those in the liver and retina. Western blot analysis showed that PEDF in the kidney was at a level comparable to that in the liver and much higher than that in the retina (FIG. 14A). Quantitative analysis using ELISA confirmed the results from Western blot analysis (FIG. 14B). There is no significant difference between PEDF levels in the kidney and those in the liver (249.21±34.45 versus 221.19±40.38 ng/mg total protein, P=0.2, n=6). The PEDF level in the retina (51.21±13.30 ng/mg total protein) was significantly lower than that in the kidney and liver (P<0.01, n=6). These results demonstrate that the kidney expresses high levels of PEDF.

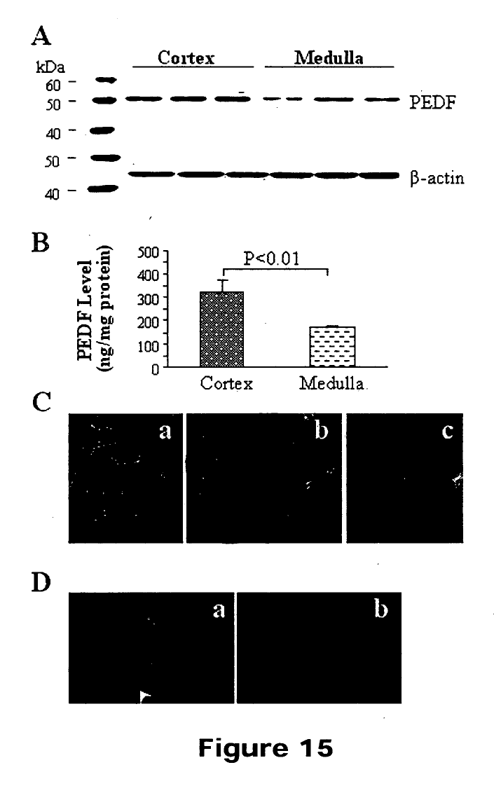

FIG. 15 illustrates high levels of PEDF in glomeruli. To determine the cellular localization of PEDF in the kidney, PEDF expression in the cortex and medulla in normal rat kidney were compared. Western blot analysis showed that PEDF levels in the renal cortex were substantially higher than that in the medulla (FIG. 15A). PEDF ELISA confirmed that the PEDF level in the cortex was 324.98±51.29 ng/mg total protein, significantly higher than that in the medulla (173.58±5.79 ng/mg total protein, P<0.01, n=6) (FIG. 15B). Immunohistochemistry using a monoclonal antibody specific for PEDF showed that PEDF was predominantly expressed in the glomeruli (FIG. 15C-a), along the parietal glomerular capsule and basement membrane (FIG. 15C-b). In the medulla, a PEDF signal was also detected at lower levels in the tubular basement membrane and interstitial tissue (FIG. 15C-c). To further identify the cellular origin of PEDF, PEDF and synaptopodin, which is accepted as a podocyte marker, were immunolabeled in the consecutive sections of the same kidney. The results showed that the distribution pattern of synaptopodin and PEDF are similar, but not identical. PEDF (FIG. 15D-a), but not synaptopodin (FIG. 15D-b), was found on the parietal glomerular capsule.

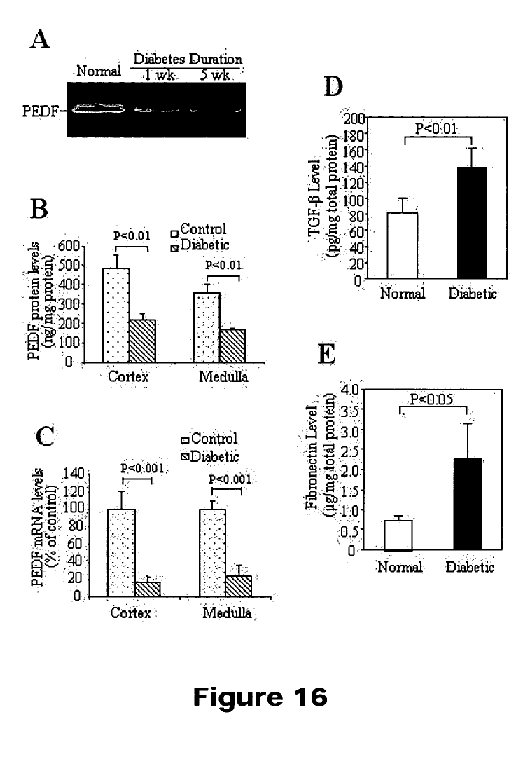

FIG. 16 illustrates decreased expression of PEDF in the kidney of STZ-induced diabetic rats. BN rats with STZ-induced diabetes developed polyuria and microalbuminuria at 1-6 weeks after the onset of diabetes, reflecting the impaired function of the glomeruli (Table 1). To determine if the change in PEDF expression is implicated in DN, PEDF levels in the kidney and serum in STZ-induced diabetic rats were compared with that in age-matched control animals. Both Western blot analysis and specific ELISA demonstrated that the PEDF protein levels were significantly decreased in diabetic cortex and medulla when compared with that in the non-diabetic control animals (FIGS. 16A & 16B). Decreased PEDF expression was also detected at the mRNA level in the diabetic cortex and medulla (FIG. 16C). In the same tissue samples, however, TGF-β and fibronectin levels were significantly increased in the diabetic kidneys (FIGS. 16D & 16E).

When compared with the age-matched non-diabetic control animals, PEDF levels in the serum were significantly decreased at the late stage of diabetes (3.54±0.83 ng/mg of total protein in 12-week diabetic rats vs 8.35±1.13 ng/mg in age-matched normal rats n=4, P<0.01), but not at the early stage in diabetic rats (7.00±2.12 ng/mg of total protein in 3-week diabetic rats vs. 7.08±1.17 ng/mg of total protein in nonormal rats, n=4, P>0.1),

TABLE 1
Microalbuminuria and General Conditions in Diabetic Rats

| Duration of | Blood Glucose | Body | | Albumin in 24-h |
|---|---|---|---|---|
| Diabetes | (mg/dl) | Weight (g) | 24-h Urine Volume (ml) | Urine (μg) |
| Normal | 106.80 ± 13.61 | 139.40 ± 6.54 | 4.13 ± 1.48 | 8.74 ± 4.20 |
| 1 wk | 498.50 ± 90.91 | 134.83 ± 10.16 | 31.25 ± 13.90 | 32.36 ± 3.95 |
| 2 wks | 504.17 ± 89.16 | 137.17 ± 17.94 | 34.25 ± 22.56 | 38.59 ± 16.36 |
| 4 wks | 462.00 ± 91.66 | 144.60 ± 16.04 | 35.25 ± 28.64 | 76.95 ± 24.50 |
| 6 wks | 510.20 ± 54.20 | 144.20 ± 21.76 | 47.00 ± 44.78 | 90.75 ± 37.45 |

*Values are expressed as mean ± SD, n = 4-6.

demonstrating that the decrease in PEDF in diabetic kidney occurs prior to the declined serum PEDF levels.

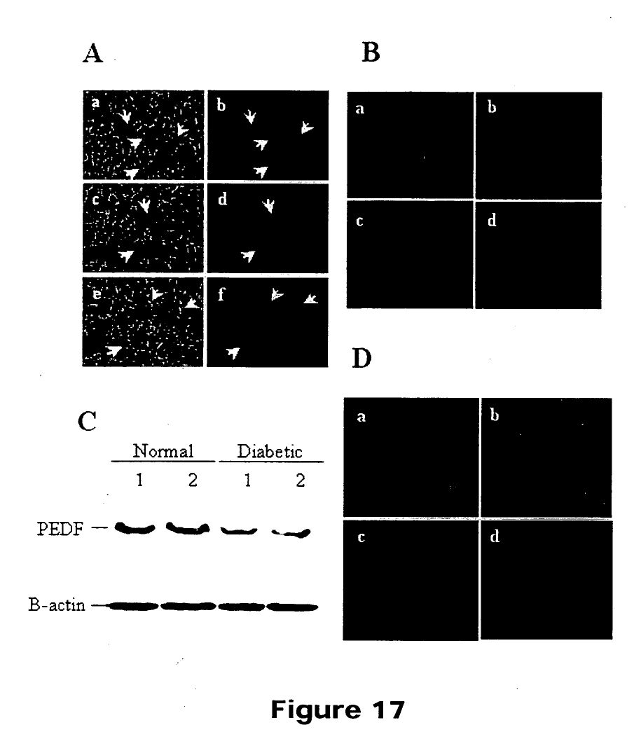

An immunohistochemistry study was performed on kidney sections from rats of one week, two week and six month diabetes and age-matched non-diabetic controls. The average body weight of diabetic rats was 20-30% lower than that of non-diabetic control animals of the same age. The results showed the decrease of PEDF in diabetic kidneys at both the early stage (one and two weeks after diabetes onset, FIGS. 17A and 17D, respectively) and the late stage (six months after diabetes onset, FIG. 17B) of diabetes. Moreover, the decrease of PEDF expression in the diabetic kidney was primarily observed in the glomeruli (FIGS. 17A, 17B and 17D), while PEDF expression was not affected in the medulla of the same diabetic kidney (FIG. 17B). Western blot analysis of PEDF in the isolated glomeruli confirmed that PEDF levels decreased in diabetic glomeruli compared with that in non-diabetic controls. At two weeks after diabetes onset, PEDF levels decreased dramatically, while no detectable podocyte loss was observed (FIG. 17D), indicating that the decrease in PEDF levels in the early stage of DN is not a result of the podocyte loss.

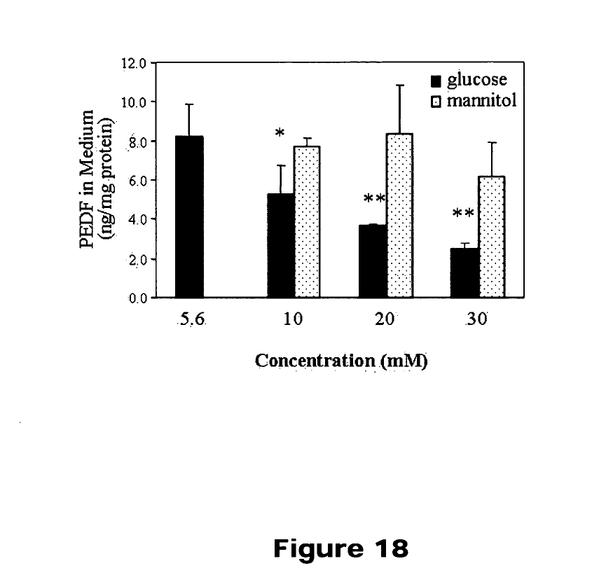

FIG. 18 illustrates high glucose decreased PEDF secretion in primary HMC. To reveal the cause for the decreased expression of PEDF in diabetic kidney, the effect of high glucose on the expression of PEDF in primary HMC was determined. HMC were cultured with different concentrations of D-glucose (Sigma. St. Louis, Mo.) for 96 h with the same concentrations of D-mannitol as osmotic controls. The PEDF secreted into the medium was quantified by ELISA. The results showed that high glucose concentrations significantly decreased PEDF secretion in HMC in a dose-dependent manner, while the mannitol had no significant effects on PEDF secretion (FIG. 18).

FIG. 19 illustrates that PEDF blocked high glucose-induced TGF-β over-expression in primary HMC. As TGF-β is recognized as one of the major mediators of the proliferation of mesangial cells and the overproduction of ECM in DN, the effects of PEDF on TGF-β secretion by HMC were also examined. Under high glucose conditions (30 mM), TGF-β secretion was significantly increased when compared with the normal glucose and mannitol controls (FIG. 19A). PEDF at concentrations of 40-160 nM significantly down-regulated TGF-β expression in a dose-dependent manner. The inhibitory effects of PEDF on TGF-β expression occurred at 24 h and lasted for at least 48 h (FIG. 19B).

FIG. 20 illustrates that PEDF blocked high glucose-induced fibronectin secretion in primary HMC. In the early stage of DN, overproduction of ECM proteins, such as fibronectin and collagen, is a major causative factor responsible for glomerular hyper-filtration and glomerular fibrosis (Weston et al., 2003). In primary HMC, exposure to high glucose (30 mM) for 48 h led to significant increases of fibronectin secretion, compared to mannitol control (FIG. 20A). At low doses (5-40 nM), PEDF decreased fibronectin secretion in a dose-dependent manner in HMC cultured in the high glucose medium (FIG. 20A). At 24 and 48 h after the addition of 40 nM PEDF, the fibronectin secretion was decreased to 23% and 18% of control, respectively (FIG. 20B).

PEDF does not affect the growth of HMC. The viability of glomerular mesangial cells and enlargement of the kidney are known as the major pathological changes in the early stage of DN. To explore whether the decrease of PEDF expression in the glomeruli contributes to the pathogenesis of DN, the effects of PEDF and high glucose on mesangial cell proliferation were studied using primary HMC. The results (data not shown) showed that neither high glucose nor PEDF of the doses from 2.5-160 nM altered -viable cell numbers in HMC, demonstrating that PEDF's effects on down-regulation of fibronectin and TGF-β are not through the inhibition of mesangial cell proliferation.

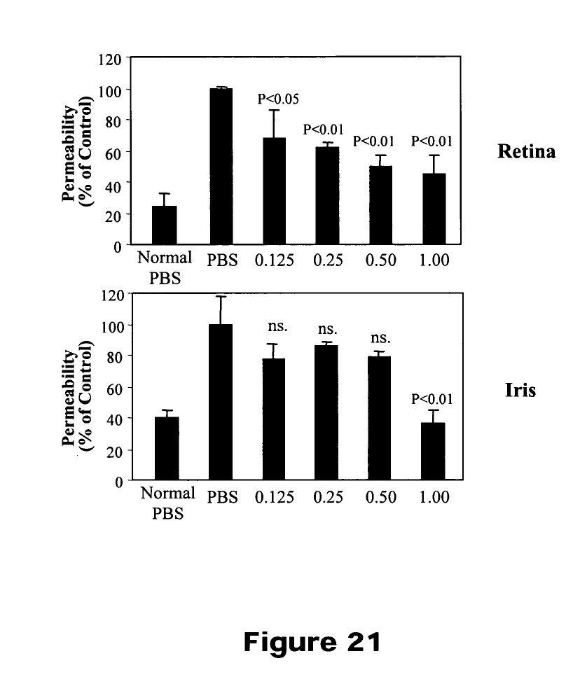

FIG. 21 illustrates that PEDF reduces retinal vascular leakage in rats with OIR. Newborn BN rats were exposed to 75% oxygen from postnatal day 7 (P7) to P12 to induce retinopathy. At age P14, the OIR rats received a single injection of 3 μl of PEDF with various concentrations to reach final doses of 0.125, 0.25, 0.50 and 1.0 μg per eye into the right eye, and the left eye received a single injection of the same volume of PBS. Two days after the injection, vascular permeability was decreased in a PEDF dose-dependent manner in the retina. At doses as low as 0.375 μg/eye, PEDF significantly reduced vascular permeability in the retina, when compared to the contralateral control (P <0.05, n =4).

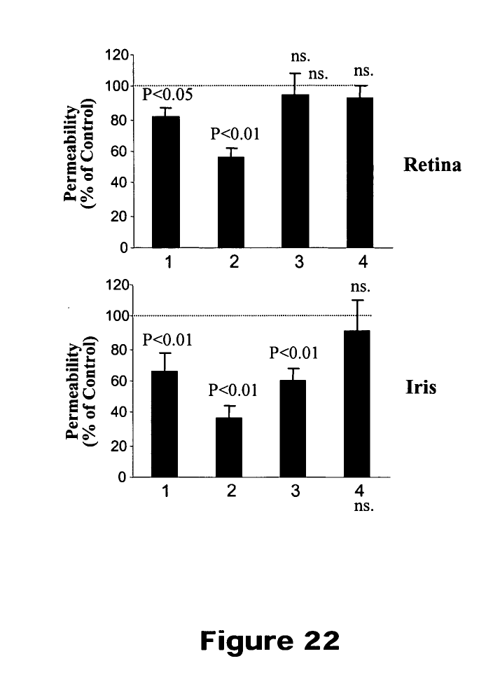

FIG. 22 illustrates the time course of the effect of PEDF on vascular permeability. PEDF was injected into the vitreous of the right eye of OIR rats at P14 (3 μg/eye) and PBS to the left eye as a control. Vascular permeability was measured at P15, P16, P17 and P18. The results demonstrate that PEDF reduces vascular permeability at days one and two after injection. By days three and four after injection of PEDF, the protein is degraded, which correlates with the diminished effect on permeability.

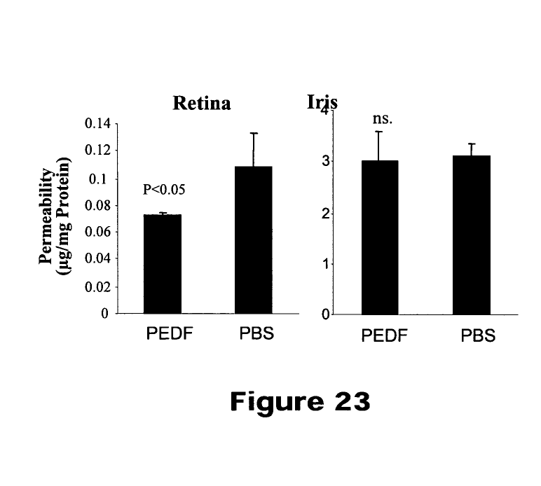

FIG. 23 illustrates that PEDF also reduces retinal vascular leakage in STZ-diabetic rats. To confirm that the PEDF-induced reduction of vascular permeability in the retina is not a result of decreased neovascularization in the OIR model, the effect of PEDF on vascular permeability was also determined in the STZ-diabetic model, which is known to have increased vascular permeability in the retina but lacks neovascularization. PEDF was injected into the vitreous space (3 μg/eye) in the right eye and PBS into the left eye of STZ-diabetic BN rats 2 wks after the induction of diabetes. Two days after the injection, the PEDF-injected eyes had a significant reduction in vascular permeability in the retina, compared to the PBS injected contralateral eyes (P<0.05, n=4). A significant difference in permeability was observed only in the retina and not in the iris, thereby indicating that PEDF also reduces vascular leakage in the STZ-diabetic model.

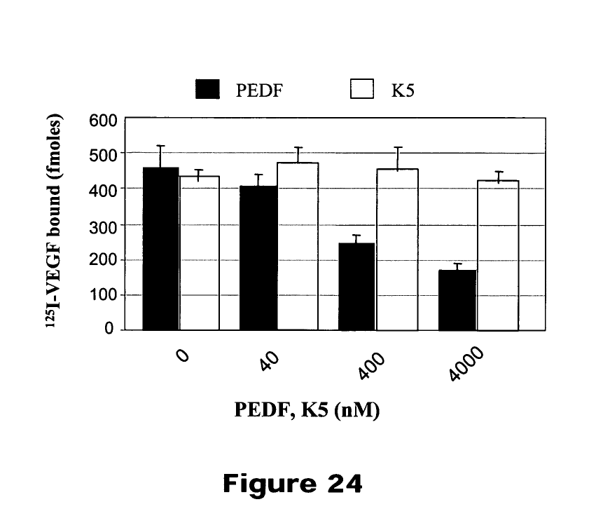

FIG. 24 illustrates that PEDF blocks VEGF binding to VEGFR. To investigate if PEDF interferes with VEGF function, binding of VEGF to its receptors on RCEC was determined. Incubation of ¹²⁵I labeled VEGF with RCEC for 2 hr resulted in significant binding of VEGF to RCEC. This binding can be blocked by the addition of excess amounts of unlabeled VEGF (data not shown). Interestingly, recombinant PEDF also competed with VEGF for RCEC binding. This competition appeared to be PEDF concentration-dependent. In contrast, plasminogen kringle 5 (K5), another potent angiogenic inhibitor, did not block VEGF binding to RCEC at the same concentrations; suggesting that PEDF may exert its vascular activity through a different mechanism than K5, which also reduces vascular leakage and down-regulates VEGF expression.

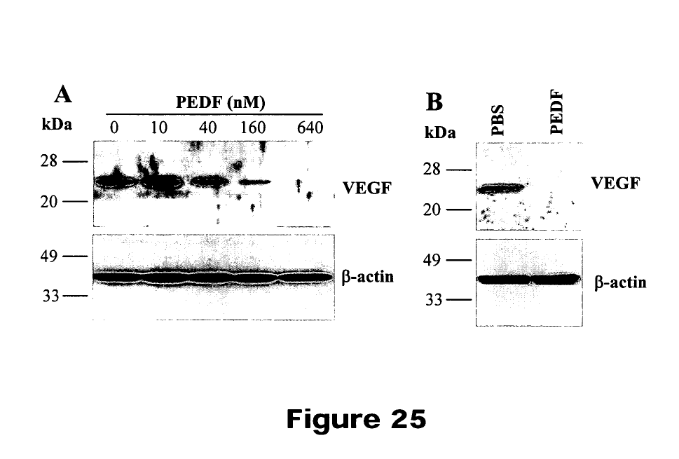

FIG. 25 illustrates that PEDF down-regulates VEGF expression in the retina of OIR rats. OIR rat retina is know to have increased VEGF levels. PEDF was injected into the vitreous of the right eye and PBS into the left eye of OIR rats at age P14. The retina was dissected at P16 for VEGF Western blot analysis. The injection of PEDF dramatically decreased retinal VEGF levels in OIR rats in a dose-dependent manner.

PEDF also down-regulates VEGF expression in endothelial cells as well as retinal Müller cells. The effect of PEDF on VEGF expression has also been determined in primary RCEC. Treatment of RCEC with various concentrations of PEDF for 24 hr. under hypoxia resulted in a PEDF concentration-dependent decrease of VEGF levels. As Müller cells are known as the major producer of VEGF in the retina, the effect of PEDF on VEGF expression was also determined in a rat Müller cell line, rMC-1. The cells were treated with various concentrations of PEDF under hypoxia for 24 hr. The VEGF was decreased in a PEDF concentration-dependent manner (data not shown). These results suggest that PEDF down-regulates VEGF expression in multiple cell types.

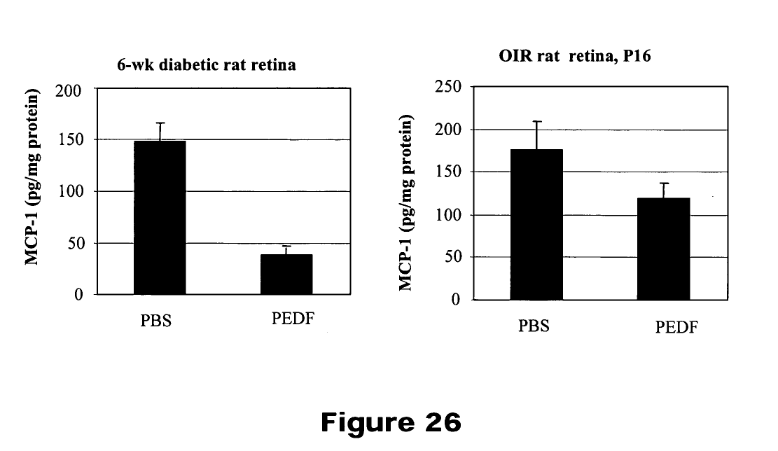

FIG. 26 illustrates that PEDF decreases MCP-1 levels in the retinas of STZ-diabetes and OIR rat models. It has been shown herein that both the STZ-diabetic and OIR retinas have increased MCP-1 levels. PEDF was injected into the vitreous of the right eye and PBS into the left eye of rats after 6 weeks of STZ-induced diabetes and rats with OIR at P16. The left eye received an injection of PBS as control. Two days following the injection, the retinas were dissected. MCP-1 was quantified using an ELISA kit and normalized by total protein concentration. The results showed that MCP-1 levels in the retina were significantly decreased by PEDF in both the models (P<0.01 in STZ rats and P<0.05 in OIR rats, n=4), thereby demonstrating that PEDF inhibits inflammation induced by diabetes and ischemia.

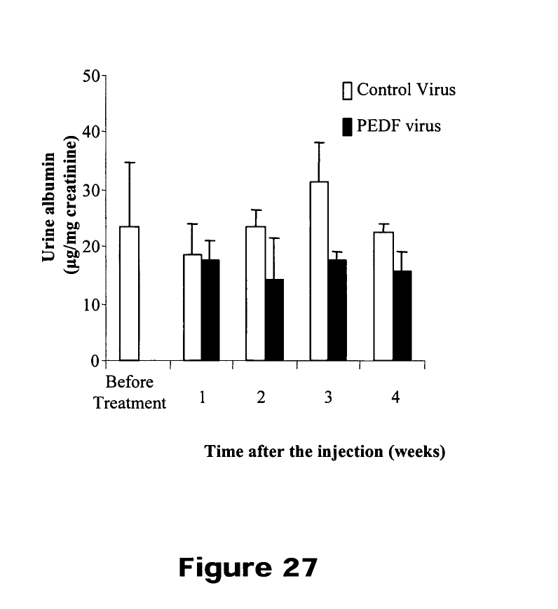

FIG. 27 illustrates that adenovirus-mediated PEDF gene delivery reduces albuminuria in diabetic rats. Two weeks after the onset of STZ-induced diabetes, rats in the treatment group received intraperitoneal injection of an adenovirus expressing PEDF, while the control rats received an injection of an adenovirus without the PEDF gene. Albumin and creatinine concentrations in the 24-h urine collected individually at one, two three and four weeks after the viral injection demonstrated that PEDF significantly reduced albuminuria in diabetic rats at two, three and four weeks after gene delivery. These experiments demonstrate that the compositions of the present invention may be administered to an animal not only directly as proteins but also in the form of an isolated nucleotide sequence from which the proteins can be expressed.

PEDF is a 50 kDa glycoprotein initially identified in human retinal pigment epithelial (RPE) cells. Its functions as a neurotrophic factor and an angiogenic inhibitor have been well studied in ocular tissues (Tombran-Tink et al., 1995; Karakousis et al., 2001; Tombran-Tink et al., 2003a; Tombran-Tink et al., 2003b; and Dawson et al., 1999; Boehm et al., 2003; Gao et al., 2001; and Gao et al., 2002). Its implication in diabetic retinopathy has been established (Tombran-Tink et al., 2003a and 2003b; Boehm et al., 2003; Gao et al., 2001; and Gao et al., 2002). The present invention reveals for the first time that PEDF may function as an endogenous inhibitor of TGF-β in the kidney, and decreased PEDF expression in diabetic kidney may contribute to the development of DN.

Previous studies have shown that PEDF is present in a variety of ocular tissues, such as the inter-photoreceptor matrix and ganglion cell layer of the retina, epithelium of the cornea and ciliary epithelium (Tombran-Tink et al., 1995; Tombran-Tink et al., 2003a; and Tombran-Tink et al., 1991). Expression of PEDF was also found in human brain and spinal cord of the neural system, and several non-neural tissues, including the liver, placenta, heart and skeletal muscle, suggesting that PEDF's function may not be limited to ocular tissues (Tombran-Tink et al., 2003). Recently, Abramson and colleagues detected the expression of PEDF in the murine kidney (Abramson et al., 2003). The present invention confirmed the expression of PEDF in rat kidney at both the mRNA and protein levels. Moreover, PEDF levels in the rat kidney, liver and retina were quantitatively compared herein. Surprisingly, the results showed that PEDF levels in the kidney are as high as that in the liver, which is considered the major source of systemic PEDF. PEDF levels in the kidney and liver are much higher than that in the retina. The high level of PEDF in the kidney underscores its significance for renal functions.

The cortex and medulla have different structures and functions in the kidney. The immunohistochemical analysis demonstrated that PEDF is predominantly present at the glomerular capsule and basement membrane in the cortex, while the PEDF signal was relatively weaker at the tubular basement membrane and the interstitial tissue in the medulla. This finding demonstrates a possible role for PEDF in the regulation of glomerular functions. This cellular localization of PEDF is different from that in Abramson's report, which mentioned that PEDF was mainly expressed in tubular epithelial cells, but not in the glomeruli of mice kidney. It is not clear what causes the disparity between the results of the present invention and that by Abramson et al. with respect to the cellular localization of PEDF in the kidney, as the figure showing the PEDF signal in the glomeruli was not presented in their paper (Abramson et al., 2003). Different species used in the experiments presented herein and in Abramson et al. may be a possible reason responsible for the disparity.

Functional studies have demonstrated that PEDF is a multifaceted factor with potent anti-angiogenesis activity and neuroprotective function (Tombran-Tink et al., 2003). PEDF inhibits endothelial cell migration induced by VEGF and fibroblast growth factor (FGF) (Tombran-Tink et al., 2003b; and Dawson et al., 1999). A recent study showed that in PEDF gene knockout mice, the microvascular density in the kidney is significantly increased compared with wild-type mice, suggesting that PEDF may play a role in the regulation of renal vasculature development and maintenance of renal homeostasis (Abramson et al., 2003).

Previous studies from both diabetic patients and animal models have demonstrated that decreased PEDF levels are involved in diabetic retinopathy (Boehm et al., 2003; Gao et al., 2001; Duh et al., 2004; and Ogata et al., 2002). Due to the close relationship between DN and diabetic retinopathy, the present invention involved determining whether PEDF levels in the kidney are also decreased in a diabetic animal model. The results showed that PEDF expression is significantly decreased in the kidney at both the mRNA and protein levels in STZ-induced diabetic rats, which have exhibited early DN changes including albuminuria and polyuria. Moreover, the decrease in PEDF levels was mainly observed in the glomeruli, while the PEDF levels in the tubular region are less affected. The location of the changes in PEDF levels in diabetic kidney provides a possible association of decreased PEDF expression with hyper-filtration of glomeruli and microalbuminuria in diabetic rats.

Diabetes is a complicated metabolic disorder and involves multiple changes in vivo. To identify the causative factor responsible for PEDF decrease in diabetic glomeruli, the effect of high glucose, a major change in diabetes, on PEDF expression in primary HMC was first determined. The results showed that high glucose significantly decreases PEDF expression, while increasing the expression of TGF-β and fibronectin in HMC, indicating that the decreased expression of PEDF in diabetic kidney may be ascribed to the direct effects of hyperglycemia. It remains to be determined how high glucose down-regulates PEDF expression in HMC.

TGF-β is a well-studied pathogenic factor of DN (Goldfarb et al., 2001; Greener, 2000; and Iglesias-de la Cruz et al., 2002). TGF-β is known to be up-regulated in diabetic kidney, which contributes to the proliferation of mesangial cell and ECM production, the major pathological changes in early DN (Goldfarb et al., 2001; Tamaki et al., 2003; Lane et al., 2001; and Lopez-Casillas, 2000). Several potential therapeutic agents exert their effects through blockade of renal TGF-β over-expression/function in diabetic kidney (Greener, 2000; Lopez-Casillas, 2000; and Chen et al., 2003). To further elucidate the function of PEDF in diabetic kidney, the effects of PEDF on TGF-β secretion in HMC were investigated. The results showed that PEDF significantly blocked high glucose-induced TGF-β over-expression, suggesting that PEDF may act as an endogenous inhibitor of TGF-β expression via a paracrine or autocrine regulation in normal kidney. This finding also suggests that decreased PEDF levels in diabetic kidney may be responsible, at least in part, for TGF-β over-expression.

One of the pathological functions of TGF-β in DN is to promote the over-production of ECM by mesangial cells, which is also closely correlated with microalbuminuria and fibrosis (Raptis et al., 2001; and American Diabetes Assoc., 2000). As mesangial cells are the major producer of ECM, primary HMC were used as a model to determine if PEDF also blocks the function of TGF-β in the induction of ECM production secretion. The results showed that high glucose concentrations (30 mM) significantly increased fibronectin secretion. PEDF blocks high glucose-induced overproduction of fibronectin in a dose-dependent manner. Further, it was tested whether the effect of PEDF on fibronectin production was through the inhibition of mesangial cell proliferation. The results showed that PEDF had no effect on the growth rate of mesangial cells. These results demonstrate that PEDF blocks ECM secretion from mesangial cells without interfering with cell proliferation.

In summary, the results provided herein above for the first time demonstrated that PEDF expression is implicated in diabetic retinopathy and DN. PEDF may play an important role in prevention of mesangial ECM overproduction, inflammation, proteinuria and pathological growth factor up-regulation in the kidney. In addition, PEDF also reduces vascular leakage and inflammation in the retina. The decreased expression or the dysfunction of PEDF may be involved in the pathogenesis of DN and DR. Therefore, PEDF will have great therapeutic potential in the treatment of various disorders involving vascular leakage, inflammation and fibrosis, such as but not limited to, diabetic retinopathy, macular edema and DN.

### Materials and Methods

Animals. Brown Norway (BN) rats were purchased from Harlan (Indianapolis, Ind.) or Charles River Laboratories (Wilmington, Mass.). Care, use, and treatment of all animals in this study were in strict agreement with the ARVO statement for the Use of Animals in Ophthalmic and Vision Research as well as the guidelines set forth in the Care and Use of Laboratory Animals by the University of Oklahoma.

Rat models of OIR and diabetes. OIR was induced by exposing newborn rats to 75% O2 as described by Smith et al. (Smith et al., 1994) with some modifications (Zhang et al., 2001). Diabetes was induced in adult rats (8 weeks of age) by an intravenous injection (for retina studies) or intraperitoneal injection (for kidney studies) of STZ (50 mg/kg in 10 mmol/L of citrate buffer, pH 4.5) (Sigma, St. Louis, Mo.) into BN rats after an overnight fasting. Control rats received an injection of citrate buffer alone. Blood glucose levels were measured at 24 h after the injection and monitored every three days thereafter. Only the animals with blood glucose concentrations higher than 350 mg/dl were considered diabetic.

Intravitreal injection of angiostatin or PEDF. Angiostatin (Angiogenesis Research Industries, Inc., Chicago, Ill.) or PEDF was reconstituted in sterile PBS and diluted to desired concentrations. For the retina studies, angiostatin or PEDF was injected into the vitreous of the right eye (3 μl/eye) of the anesthetized rats through the pars plana using a glass capillary, while the left eye received the same volume of sterile PBS as the control.

Measurement of vascular permeability. Vascular permeability was quantified by measuring albumin leakage from blood vessels into the retina and iris using the Evans blue method following a documented protocol (Xu et al., 2001) with minor modifications (Gao et al., 2003).

Western blot analysis. Angiostatin Western blot analysis was performed using a monoclonal anti-angiostatin antibody (Chemicon Inc., Temecula, Calif.), and PEDF Western blot analysis was performed using a monoclonal anti-PEDF antibody (Chemicon Inc., Temecula, Calif.), both as described previously (Gao et al., 2002). VEGF Western blot analysis was performed using an antibody specific for VEGF (Santa Cruz Biotechnology, Inc., Santa Cruz, Calif.) as described previously (C. saky et al., 2001). Immunohistochemistry of VEGF was carried out following a documented protocol (Rohrer et al., 1995).

For the Western blots, kidney tissue was homogenized and centrifuged at 4° C. The protein concentration in the supernatant was measured with the BioRad DC protein assay (BioRad Laboratories, Hercules, Calif.). 50 μg of protein from each sample was blotted by anti-angiostatin antibody (R&D Systems, Minneapolis, Minn.). The same membranes were stripped and reblotted by anti-VEGF and anti-ICAM-1 (Santa Cruz Biotechnology, Santa Cruz, Calif.) antibodies.

Cell culture. Primary human glomerular mesangial (HMC) cells were purchased from Cambrex Bio Science Walkersville, Inc. (Walkersville, Md.). The cells were cultured in Mesangial cell basal medium (Cambrex) with 0.1% GA-1000 (Cambrex) and 5% fetal bovine serum (FBS) at 37° C. in a humidified 5% CO₂ atmosphere. Cells of passages from 6 to 10 were used in the experiments. After reaching 80% confluence, cells were exposed to medium with 1% FBS for 12 h before the treatments with high glucose or PEDF or angiostatin.

Isolation of glomeruli. Rats were deeply anesthetized and the kidneys were immediately removed. The cortex was excised, cut into fine fragments, and homogenized. After passed through consecutive stainless steel screens of 150 and 75 μm pore size, the glomeruli were suspended in 1× PBS and collected by centrifuge at 2000× g for 3 min.

Quantitative real-time reversetranscription (RT)-PCR. The total RNA was isolated from tissues using the RNeasy Mini-isolation Kit following the manufacturer's protocol (Qiagen, Santa Clarita, Calif.). Primers (PEDF-F, 5′-aagtcatatgggaccaggccc-3′, PEDF-R, 5-′ttacccactgccccttgaagt-3′) were designed from mRNA sequences spanning more than 1 kb introns using the Primer 3 software (http://www-genome.wi.mit.edu/cgi-bin/primer/primer3.cgi/). RT reaction used 1.0 μg total RNA, oligo-dT primer and MuLV reverse transcriptase in a final volume of 20 ∞l and conducted at 42° C. for 60 min, followed by a denaturation at 95° C. for 5 min. The real-time PCR used 1 μl of the RT product and 3 pmol of primers and was performed using GeneAmp® RNA PCR kit and SYBR® Green PCR Master Mix (Applied Biosystems). The PCR mix was denatured at 95° C. for 10 min, followed by 40 cycles of melting at 95° C. for 15 sec and elongation at 60° C. for 60 sec. Fluorescence changes were monitored after each cycle. Amplicon size and reaction specificity were confirmed by 2.5% agarose gel electrophoresis. All reactions were performed in triplicate. The average C_{T} (threshold cycle) of fluorescence units was used to analyze the mRNA levels. The PEDF or MMP-2 mRNA levels were normalized by 18s ribosomal RNA levels. Quantification was calculated as: mRNA levels (percent of control)=2Δ(Δ^{C}_{T}) with ΔC_{T}=C_{T, PEDF}−C_{T, GAPDH} and Δ(ΔC_{T})=ΔC_{T, normal sample}−ΔC_{T, STZ-diabetic sample}.

Immunohistochemistry study. Immunohistochemistry was performed on frozen tissue sections. Briefly, the sections were blocked with solution containing 3% BSA (Sigma, St. Louis, Mo.) and 5% rabbit serum (Jackson Immunoresearch, PA). After incubation with 1:800 dilution of an anti-PEDF antibody (Chemicon, CA) or an anti-synaptopodin antibody (Biodesign, ME) for 1 h, the sections were thoroughly washed and incubated with 1:200 FITC-conjugated rabbit anti-mouse antibody (Jackson Immunoresearch, PA) for 1 h. After extensive washing, the sections were visualized and photographed under a fluorescent microscope (Olympus, Humburg, Germany) and confocal microscope (Leica, Mannheim, Germany).

MMP-2 activity assay by gelatin zymography. Gelatinolytic activity of MMP-2 was analyzed by gelatin zymography. 15 μg of tissue extracts or 20 μl of cell culture medium were applied to a pre-cast 10% polyacrylamide gel copolymerized with 1 mg/ml gelatin (BioRad Laboratories, Hercules, Calif.). After electrophoresis, the gel was incubated with renaturation buffer (BioRad) for 1 h, followed by incubation with development buffer (BioRad) overnight. The gel was stained with simple blue staining solution (BioRad) and photographed with Imager (Syngene, Cambridge, UK).

PEDF ELISA. The protein concentration was measured with the BioRad DC protein assay (BioRad Laboratories, Hercules, Calif.). The amounts of PEDF in the tissue extracts, serum and the conditioned medium were determined using a commercial ELISA kit specific for PEDF (Chemicon Inc., Temecula, Calif.) according to the manufacturer's instructions. Briefly, the tissue samples were homogenized in 1× PBS and centrifuged (TL; Beckman) at 50,000 rpm (Rotor type: TLA 100.3) for 20 min at 4° C. The serum or tissue extract was incubated with 8 M urea (Sigma, St. Luis, Mo.) for 1 h and diluted 1:200 before being applied to the plate. After incubation at 37° C. for 1 h and the extensive washing, the plate was incubated with 100 μL of a biotinylated mouse anti-PEDF antibody for 1 h, followed by incubation with 100 μL streptavidin peroxidase conjugate for 1 h. After the addition of TMB/E for 5-10 min, the plate was read immediately at 450 nm by a Wallac-Victor3TM 1420 microplate reader (Perkin-Elmer Wallac, Inc.). For standardization, the PEDF concentration was normalized by the total protein concentration in the samples.

ELISA specific for TGF-β. TGF-β protein was quantified using the commercial Quantikine TGF-β1 ELISA Kit (R&D Systems, Minneapolis, Minn.). Briefly, the tissue extracts were activated with 1 N HCl for 10 min, followed by neutralization with 1.2 N NaOH. The activated samples were applied to the plate pre-coated with soluble type II receptor and incubated at room temperature for 3 h. After extensive washing, HRP-conjugated anti-TGF-β antibody was added and incubated for another 1.5 h. Then the chromogen was added and the plate was read at 450 nm. The results were expressed as picograms per milligram of total protein.

ELISA specific for fibronectin. Fibronectin protein was quantified by competitive. sandwich ELISA (Assaypro, Winfield, Mo.). Briefly, samples were diluted and applied to the plate coated with anti-fibronectin antibody, and the same amount of biotin-labeled fibronectin was immediately added to the wells. After incubation for 1.5 h and extensive washing, the HRP-conjugated streptavidin was added to the wells and incubated for 30 min. Then the chromogen was added and the plate was read at 450 nm. The results were expressed as micrograms per milligram of total protein.

Smad nuclear translocation assay. Primary human mesangial cells were seeded on 4-chamber slides (Nalge Nunc International Corp., Naperville, Ill.). After reaching 80% confluence, cells were exposed to the medium with 1% serum for 12 h and treated with 2.5 ng/ml TGF-β with or without 160 nM PEDF for 1 hr. Cells were immediately washed with 1× PBS and fixed with 4% paraformaldehyde for 15 min. After incubation with blocking buffer containing 3% BSA, 5% normal donkey serum and 0.2% Triton X-100 for 30 min, the cells were incubated with anti-smad2/3 antibody (1:200, Upstate USA, Inc., IL) for 2 hr at room temperature and washed with 0.05 M Tris-HCl buffer with 0.15 NaCl, pH=7.5. The cells were then incubated with cy3-conjugated donkey anti-rabbit antibody for 1 hr at room temperature. After extensive washing, the slides were visualized and photographed under a fluorescent microscope (Olympus, Humburg, Germany) and confocal microscope (Leica, Mannheim, Germany).

Cell proliferation assay. The tetrazolium dye-reduction assay (MTT; 3-[4,5-dimethylthiazol-2-yl]-2,5-diphenyl tetrazolium bromide; Sigma-Aldrich) was used to determine cell survival and proliferation rate according to the manufacturer's protocol. Briefly, the primary HMC were seeded in 12-well plates at a density of 5×10⁴ cells/well, 24 h before the treatments. Then the growth medium was replaced by a medium containing 1% FBS with desired agents such as PEDF or angiostatin (at concentrations of 2.5-160 nM). After incubation for 72 h, cells were washed with PBS, and 100 μL/well MTT solution was added and incubated at 37° C. for 4 h. The formazan crystals that formed were dissolved by incubation with dimethyl sulfoxide (DMSO; 1 ml/well) overnight. Absorption was measured at 550 nm, and the number of viable cells was calculated according to standard curve. Experiments were performed in triplicate.

Evaluation of rat microalbuminuria. The 24-h urine collected from each diabetic rat and age-matched control was centrifuged at 2000× g for 10 min. The concentration of albumin in the supernatant was measured by ELISA according to the manufacturer's protocol (Bethyl Laboratories Inc, Montgomery, Tex.). The total amount of albumin in 24-h urine was calculated accordingly.

Statistical analysis. Statistical analysis employed the Student's t test. The paired t test was used for comparison of the angiostatin-injected eye with the PBS-injected contralateral controls from the same animal, while the unpaired test was used for inter-animal comparison. Statistical difference was considered significant at a P value of less than 0.05.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the composition, methods and in the steps or in the sequence of steps of the method described herein without departing from the concept, spirit and scope of the invention. More specifically, it will be apparent that certain agents and peptides which are both chemically and physiologically related may be substituted for the agents and peptides described herein while the same or similar results would be achieved. All such similar substitutes and modifications apparent to those skilled in the art are deemed to be within the spirit, scope and concept of the invention as defined by the appended claims.

### References

The following references, to the extent that they provide exemplary procedural or other details supplementary to those set forth herein, are specifically incorporated herein by reference.
1. Abramson L P, Stellmach V, Doll J A, Cornwell M, Arensman R M, Crawford S E: Wilms' tumor growth is suppressed by antiangiogenic pigment epithelium-derived factor in a xenograft model. J Pediatr Surg 38: 336-342; discussion 336-342, 2003.
2. Adamis, A. P., Miller, J. W., Bernal, M. T., D'Amico, D. J., Folkman, J., Yeo, T. K., and Yeo, K. T. 1994. Increased vascular endothelial growth factor levels in the vitreous of eyes with proliferative diabetic retinopathy. Am. J. Ophthalmol. 118:445-450.
3. Aiello, L. P., Avery, R. L., Arrigg, P. G., Keyt, B. A., Jampel, H. D., Shah, S. T., Pasquale, L. R., Thieme, H., Iwamoto, M. A., and Park, J. E. 1994. Vascular endothelial growth factor in ocular fluid of patients with diabetic retinopathy and other retinal disorders. New Eng. J. Med. 331:1480-1487.
4. Aiello L P: Vascular endothelial growth factor and the eye: biochemical mechanisms of action and implications for novel therapies. Ophthalmic Res 29: 354-362, 1997.
5. Aiello, L. P., Bursell, S. E., Clermont, A., Duh, E., Ishii, H., Takagi, C., Mori, F., Ciulla, T. A., Ways, K., Jirousek, M., et al. 1997. Vascular endothelial growth factor-induced retinal permeability is mediated by protein kinase C in vivo and suppressed by an orally effective beta-isoform-selective inhibitor. Diabetes 46:1473-1480.
6. Aiello, L. P., and Wong, J. S. 2000. Role of vascular endothelial growth factor in diabetic vascular complications. Kidney Intl. -Suppl. 77:S113-119.
7. Amann, B., Tinzmann, R., and Angelkort, B. 2003. ACE inhibitors improve diabetic nephropathy through suppression of renal MCP-1. Diabetes Care 26:2421-2425.
8. American Diabetes Association: Diabetic nephropathy. Diabetes Care. 23: S69-72, 2000.
9. Anonymous: Effect of intensive therapy on the development and progression of diabetic nephropathy in the Diabetes Control and Complications Trial. The Diabetes Control and Complications (DCCT) Research Group. Kidney Int 47: 1703-1720, 1995.
10. Anonymous: Retinopathy and nephropathy in patients with type 1 diabetes four years after a trial of intensive therapy. The Diabetes Control and Complications Trial/Epidemiology of Diabetes Interventions and Complications Research Group.[erratum appears in N Engl J Med May 4, 2000;342(18):1376]. New Engl J Med 342: 381-389, 2000.
11. Antonetti, D. A., Barber, A. J., Khin, S., Lieth, E., Tarbell, J. M., and Gardner, T. W. 1998. Vascular permeability in experimental diabetes is associated with reduced endothelial occludin content: vascular endothelial growth factor decreases occludin in retinal endothelial cells. Penn State Retina Research Group. Diabetes 47:1953-1959.
12. Antonetti, D. A., Lieth, E., Barber, A. J., and Gardner, T. W. 1999. Molecular mechanisms of vascular permeability in diabetic retinopathy. Sem. Ophthalmol. 14:240-248.
13. Basile, D. P., Fredrich, K., Weihrauch, D., Hattan, N., and Chilian, W. M. 2004. Angiostatin and matrix metalloprotease expression following ischemic acute renal failure. American Journal of Physiology Renal Fluid & Electrolyte Physiology 286.
14. Battegay, E. J. 1995. Angiogenesis: mechanistic insights, neovascular diseases, and therapeutic prospects. J. Mol. Med. 73:333-346.
15. Benelli, R., Morini, M., Brigati, C., Noonan, D. M., and Albini, A. 2003. Angiostatin inhibits extracellular HIV-Tat-induced inflammatory angiogenesis. International Journal of Oncology 22:87-91.
16. Boehm B O, Lang G, Volpert O, Jehle P M, Kurkhaus A, Rosinger S, Lang G K, Bouck N: Low content of the natural ocular anti-angiogenic agent pigment epithelium-derived factor (PEDF) in aqueous humor predicts progression of diabetic retinopathy. Diabetologia. 46: 394-400, 2003.
17. Bolton, S. J., Anthony, D. C., and Perry, V. H. 1998. Loss of the tight junction proteins occludin and zonula occludens-1 from cerebral vascular endothelium during neutrophil-induced blood-brain barrier breakdown in vivo. Neuroscience. 86:1245-1257.
18. Bortoloso, E., Del Prete, D., Gambaro, G., Dalla Vestra, M., Sailer, A., Baggio, B., Antonucci, F., Fioretto, P., and Anglani, F. 2001. Vascular endothelial growth factor (VEGF) and VEGF receptors in diabetic nephropathy: expression studies in biopsies of type 2 diabetic patients. Renal Failure. 23:483-493.
19. Bresnick, G. H. 1986. Diabetic macular edema. A review. Ophthalmology 93:989-997.
20. C.saky, K. G., Baffi, J., Byrnes, G., and Cousins, S. W. 2001. Strain specific phenotypic variation in VEGF-induced choroidal neovascularization in the rat. Invest. Ophthalmol. Vis. Sci. 42:s227.
21. Cao, Y. 1999. Therapeutic potentials of angiostatin in the treatment of cancer. Haematologica. 84:643-650.
22. Cao, Y., and Xue, L. 2004. Angiostatin. Seminars in Thrombosis and Hemostasis 30:83-93.
23. Chen, S., Jim, B., and Ziyadeh, F. N. 2003. Diabetic nephropathy and transforming growth factor-beta: transforming our view of glomerulosclerosis and fibrosis build-up. Seminars in Nephrology. 23:532-543.
24. Chen S, Iglesias-de la Cruz M C, Jim B, Hong S W, Isono M, Ziyadeh F N: Reversibility of established diabetic glomerulopathy by anti-TGF-beta antibodies in db/db mice. Biochem Biophys Res Commun 300: 16-22, 2003.
25. Chiarelli F, Santilli F, Mohn A: Role of growth factors in the development of diabetic complications. Horm Res 53: 53-67, 2000.
26. Ciulla, T. A., Danis, R. P., and Harris, A. 1998. Age-related macular degeneration: a review of experimental treatments. Surv. Ophthalmol. 43:134-146.
27. Claesson-Welsh, L., Welsh, M., Ito, N., Anand-Apte, B., Soker, S., Zetter, B., O'Reilly, M., and Folkman, J. 1998. Angiostatin induces endothelial cell apoptosis and activation of focal adhesion kinase independently of the integrin-binding motif RGD. Proc Natl Acad Sci USA 95:5579-5583.
28. Cooper M E: Interaction of metabolic and haemodynamic factors in mediating experimental diabetic nephropathy. Diabetologia. 44: 1957-1972, 2001.
29. Cunha-Vaz, J. G., Gray, J. R., Zeimer, R. C., Mota, M. C., Ishimoto, B. M., and Leite, E. 1985. Characterization of the early stages of diabetic retinopathy by vitreous fluorophotometry. Diabetes 34:53-59.
30. Dawson D W, Volpert O V, Gillis P, Crawford S E, Xu H, Benedict W, Bouck N P: Pigment epithelium-derived factor: a potent inhibitor of angiogenesis. Science 285: 245-258, 1999.
31. de Vriese, A. S., Tilton, R. G., Elger, M., Stephan, C. C., Kriz, W., and Lameire, N. H. 2001.

Antibodies against vascular endothelial growth factor improve early renal dysfunction in experimental diabetes. Journal of the American Society of Nephrology. 12:993-1000.
32. Del Maschio, A., Zanetti, A., Corada, M., Rival, Y., Ruco, L., Lampugnani, M. G., and Dejana, E. 1996. Polymorphonuclear leukocyte adhesion triggers the disorganization of endothelial cell-to-cell adherens junctions. J. Cell Biol. 135:497-510.
33. Doll J A, Stellmach V M, Bouck N P, Bergh A R, Lee C, Abramson L P, Cornwell M L, Pins M R, Borensztajn J, Crawford S E: Pigmentepithelium-derived factor regulates the vasculature and mass of the prostate and pancreas. Nat Med 9: 774-780, 2003.
34. Drixler, T. A., Rinkes, I. H., Ritchie, E. D., Treffers, F. W., van Vroonhoven, T. J., Gebbink, M. F., and Voest, E. E. 2001. Angiostatin inhibits pathological but not physiological retinal angiogenesis. Investigative Ophthalmology & Visual Science. 42:3325-3330.
35. Duh E J, Yang H S, Haller J A, De Juan E, Humayun M S, Gehlbach P, Melia M, Pieramici D, Harlan J B, Campochiaro P A, Zack D J: Vitreous levels of pigment epithelium-derived factor and vascular endothelial growth factor: implications for ocular angiogenesis. Am J Ophthalmol 137: 668-674, 2004.
36. Dvorak H F, Brown L F, Detmar M, Dvorak A M: Vascular permeability factor/vascular endothelial growth factor, microvascular hyperpermeability, and angiogenesis. Am J Pathol 146: 1029-1039, 1995.
37. Eliceiri, B. P., and Cheresh, D. A. 1999. The role of alpha v integrins during angiogenesis: insights into potential mechanisms of action and clinical development. J. Clin. Invest. 103:1227-1230.
38. Eliceiri, B. P., Klemke, R., Stromblad, S., and Cheresh, D. A. 1998. Integrin alphavbeta3 requirement for sustained mitogen-activated protein kinase activity during angiogenesis. J. Cell Biol. 140:1255-1263.
39. Ellis D, Forrest K Y, Erbey J, Orchard T J: Urinary measurement of transforming growth factor-beta and type IV collagen as new markers of renal injury: application in diabetic nephropathy. Clin Chem 44: 950-956, 1998.
40. Flyvbjerg, A. 2000. Putative pathophysiological role of growth factors and cytokines in experimental diabetic kidney disease. Diabetologia. 43:1205-1223.
41. Flyvbjerg, A., Dagnaes-Hansen, F., De Vriese, A. S., Schrijvers, B. F., Tilton, R. G., and Rasch, R. 2002. Amelioration of long-term renal changes in obese type 2 diabetic mice by a neutralizing vascular endothelial growth factor antibody. Diabetes. 51:3090-3094.
42. Gambaro G, Ceol M, Del Prete D, D'Angelo A: GLUT-1 and TGF-beta: the link between hyperglycaemia and diabetic nephropathy.[comment]. Nephrol Dial Transplant 15: 1476-1477, 2000.
43. Gao G, Li Y, Zhang D, Gee S, Crosson C, Ma J: Unbalanced expression of VEGF and PEDF in ischemia-induced retinal neovascularization. FEBS Lett 489: 270-276, 2001.
44. Gao G, Li Y, Gee S, Dudley A, Fant I, Crosson C, Ma J-X: Down-regulation of VEGF and up-regulation of PEDF: a possible mechanism for the anti-angiogenic activity of plasminogen kringle 5. J Biol Chem 277: 9492-9497, 2002.
45. Gao, G., Shao, C., Zhang, S. X., Dudley, A., Fant, J. and Ma, J.-x. (2003) Diabetologia 46, 689-698.
46. Goldfarb, S., and Ziyadeh, F. N. 2001. TGF-beta: a crucial component of the pathogenesis of diabetic nephropathy. Transactions of the American Clinical & Climatological Association. 112:27-32; discussion 33.
47. Greener, M. 2000. Targeting TGF could counter diabetic nephropathy. Molecular Medicine Today. 6:376.
48. Guler, S., Cakir, B., Demirbas, B., Yonem, A., Odabasi, E., Onde, U., Aykut, O., and Gursoy, G. 2002. Plasma soluble intercellular adhesion molecule 1 levels are increased in type 2 diabetic patients with nephropathy. Hormone Research 58:67-70.
49. Ha, H., Yu, M. R., Choi, Y. J., Kitamura, M., and Lee, H. B. 2002. Role of high glucose-induced nuclear factor-kappaB activation in monocyte chemoattractant protein-1 expression by mesangial cells. Journal of the American Society of Nephrology 13:894-902.
50. Hammes, H. P., Lin, J., Bretzel, R. G., Brownlee, M., and Breier, G. 1998. Upregulation of the vascular endothelial growth factor/vascular endothelial growth factor receptor system in experimental background diabetic retinopathy of the rat. Diabetes. 47:401-406.
51. Iglesias-de la Cruz M C, Ziyadeh F N, Isono M, Kouahou M, Han D C, Kalluri R, Mundel P, Chen S: Effects of high glucose and TGF-beta1 on the expression of collagen IV and vascular endothelial growth factor in mouse podocytes. Kidney Int. 62: 901-913, 2002.
52. Janssen, U., Sowa, E., Marchand, P., Floege, J., Phillips, A. O., and Radeke, H. H. 2002. Differential expression of MCP-1 and its receptor CCR2 in glucose primed human mesangial cells. Nephron 92:797-806.
53. Karakousis P C, John S K, Behling K C, Surace E M, Smith J E, Hendrickson A, Tang W X, Bennett J, Milam A H: Localization of pigment epithelium derived factor (PEDF) in developing and adult human ocular tissues. Mol Vis 7: 154-163, 2001.
54. Kato, S., Luyckx, V. A., Ots, M., Lee, K. W., Ziai, F., Troy, J. L., Brenner, B. M., and MacKenzie, H. S. 1999. Renin-angiotensin blockade lowers MCP-1 expression in diabetic rats. Kidney International 56:1037-1048.
55. Khamaisi, M., Schrijvers, B. F., De Vriese, A. S., Raz, I., and Flyvbjerg, A. 2003. The emerging role of VEGF in diabetic kidney disease. Nephrol Dial Transplant 18:1427-1430.
56. Kim B S, Goligorsky M S: Role of VEGF in kidney development, microvascular maintenance and pathophysiology of renal disease. Korean J Intern Med 18: 65-75, 2003.
57. Kirsch, M., Strasser, J., Allende, R., Bello, L., Zhang, J., and Black, P. M. 1998. Angiostatin suppresses malignant glioma growth in vivo. Cancer Research 58:4654-4659.
58. Lai, C. C., Wu, W. C., Chen, S. L., Xiao, X., Tsai, T. C., Huan, S. J., Chen, T. L., Tsai, R. J., and Tsao, Y. P. 2001. Suppression of choroidal neovascularization by adeno-associated virus vector expressing angiostatin. Invest. Ophthalmol. Vis. Sci. 42:2401-2407.
59. Lane P H, Snelling D M, Langer W J: Streptozocin diabetes elevates all isoforms of TGF-beta in the rat kidney. Int J Exp Diabetes Res 2: 55-62, 2001.
60. Leehey, D. J., Singh, A. K., Alavi, N., and Singh, R. 2000. Role of angiotensin II in diabetic nephropathy. Kidney International Supplement 77:S93-98.
61. Liu H, Ren J G, Cooper W L, Hawkins C E, Cowan M R, Tong P Y: Identification of the antivasopermeability effect of pigment epithelium-derived factor and its active site. Proc Natl Acad Sci USA 101: 6605-6610, 2004.
62. Lopes de Faria, J. M., Jalkh, A. E., Trempe, C. L., and McMeel, J. W. 1999. Diabetic macular edema: risk factors and concomitants. Acta Ophthalmol. Scand. 77:170-175.
63. Lopez-Casillas F: [Transforming growth factor-beta (TGF-β), keystone in diabetic nephropathy]. Rev Invest Clin 52: 487-490, 2000.
64. Makino, H., Sugimoto, H., and Shikata, K. 1999. [Diabetic nephropathy—recent advances in its mechanism and treatment]. Nippon Rinsho Japanese Journal of Clinical Medicine 57:590-600.
65. Meneses, P. I., Hajjar, K. A., Berns, K. I., and Duvoisin, R. M. 2001. Recombinant angiostatin prevents retinal neovascularization in a murine proliferative retinopathy model. Gene Therapy 8:646-648.
66. Milanini, J., Vinals, F., Pouyssegur, J., and Pages, G. 1998. p42/p44 MAP kinase module plays a key role in the transcriptional regulation of the vascular endothelial growth factor gene in fibroblasts. J. Biol. Chem. 273:18165-18172.
67. Miyamoto, K., Khosrof, S., Bursell, S. E., Moromizato, Y., Aiello, L. P., Ogura, Y., and Adamis, A. P. 2000. Vascular endothelial growth factor (VEGF)-induced retinal vascular permeability is mediated by intercellular adhesion molecule-1 (ICAM-1). Am. J. Pathol. 156:1733-1739.
68. Miyamoto, K., Khosrof, S., Bursell, S. E., Rohan, R., Murata, T., Clermont, A. C., Aiello, L. P., Ogura, Y., and Adamis, A. P. 1999. Prevention of leukostasis and vascular leakage in streptozotocin-induced diabetic retinopathy via intercellular adhesion molecule-1 inhibition. Proc. Natl. Acad. Sci. USA. 96:10836-10841.
69. Mogyorosi A, Kapoor A, Isono M, Kapoor S, Sharma K, Ziyadeh F N: Utility of serum and urinary transforming growth factor-beta levels as markers of diabetic nephropathy. Nephron 86: 234-235, 2000.
70. Mori K, Duh E, Gehlbach P, Ando A, Takahashi K, Pearlman J, Yang H S, Zack D J, Ettyreddy D, Brough D E, Wei L L, Campochiaro P A: Pigment epithelium-derived factor inhibits retinal and choroidal neovascularization. J Cell Physiol 188: 253-263, 2001.
71. Moriwaki, Y., Yamamoto, T., Shibutani, Y., Aoki, E., Tsutsumi, Z., Takahashi, S., Okamura, H., Koga, M., Fukuchi, M., and Hada, T. 2003. Elevated levels of interleukin-18 and tumor necrosis factor-alpha in serum of patients with type 2 diabetes mellitus: relationship with diabetic nephropathy.[see comment]. Metabolism: Clinical & Experimental 52:605-608.
72. Moss, S. E., Klein, R., and Klein, B. E. 1998. The 14-year incidence of visual loss in a diabetic population. Ophthalmology. 105:998-1003.
73. Murata, T., Nakagawa, K., Khalil, A., Ishibashi, T., Inomata, H., and Sueishi, K. 1996. The relation between expression of vascular endothelial growth factor and breakdown of the blood-retinal barrier in diabetic rat retinas. Lab. Invest. 74:819-825.
74. Nishioka, H., Kanauchi, M., and Dohi, K. 2001. Role of monocyte chemotactic peptide 1 in diabetic nephropathy. Nephron 88:189-190.
75. Ng D P, Warram J H, Krolewski A S: TGF-beta 1 as a genetic susceptibility locus for advanced diabetic nephropathy in type 1 diabetes mellitus: an investigation of multiple known DNA sequence variants. Am J Kidney Dis 41: 22-28, 2003.
76. Ogata N, Nishikawa M, Nishimura T, Mitsuma Y, Matsumura M: Unbalanced vitreous levels of pigment epithelium-derived factor and vascular endothelial growth factor in diabetic retinopathy. Am J Ophthalmol 134: 348-353, 2002.
77. Okada, S., Shikata, K., Matsuda, M., Ogawa, D., Usui, H., Kido, Y., Nagase, R., Wada, J., Shikata, Y., and Makino, H. 2003. Intercellular adhesion molecule-1-deficient mice are resistant against renal injury after induction of diabetes. Diabetes 52:2586-2593.
78. O'Reilly, M. S., Holmgren, L., Shing, Y., Chen, C., Rosenthal, R. A., Moses, M., Lane, W. S., Cao, Y., Sage, E. H., and Folkman, J. 1994. Angiostatin: a novel angiogenesis inhibitor that mediates the suppression of metastases by a Lewis lung carcinoma. Cell 79:315-328.
79. O'Reilly, M. S., Holmgren, L., Chen, C., and Folkman, J. 1996. Angiostatin induces and sustains dormancy of human primary tumors in mice. Nat Med 2:689-692.
80. Pages, G., Milanini, J., Richard, D. E., Berra, E., Gothie, E., Vinals, F., and Pouyssegur, J. 2000. Signaling angiogenesis via p42/p44 MAP kinase cascade. Ann. N. Y. Acad. Sci. 902:187-200.
81. Pe'er, J., Shweiki, D., Itin, A., Hemo, I., Gnessin, H., and Keshet, E. 1995. Hypoxia-induced expression of vascular endothelial growth factor by retinal cells is a common factor in neovascularizing ocular diseases. Lab. Invest. 72:638-645.
82. Pfeiffer A, Middelberg-Bisping K, Drewes C, Schatz H: Elevated plasma levels of transforming growth factor-beta 1 in NIDDM. Diabetes Care 19: 1113-1117, 1996.
83. Pierce, E. A., Avery, R. L., Foley, E. D., Aiello, L. P., and Smith, L. E. 1995. Vascular endothelial growth factor/vascular permeability factor expression in a mouse model of retinal neovascularization. Proc. Natl. Acad. Sci. USA. 92:905-909.
84. Poulaki, V., Qin, W., Joussen, A. M., Hurlbut, P., Wiegand, S. J., Rudge, J., Yancopoulos, G. D., and Adamis, A. P. 2002. Acute intensive insulin therapy exacerbates diabetic blood-retinal barrier breakdown via hypoxia-inducible factor-1alpha and VEGF. J. Clin. Invest. 109:805-815.
85. Qaum, T., Xu, Q., Joussen, A. M., Clemens, M. W., Qin, W., Miyamoto, K., Hassessian, H., Wiegand, S. J., Rudge, J., Yancopoulos, G. D., et al. 2001. VEGF-initiated blood-retinal barrier breakdown in early diabetes. Invest. Ophthalmol. Vis. Sci. 42:2408-2413.
86. Raptis, A. E., and Viberti, G. 2001. Pathogenesis of diabetic nephropathy. Experimental & Clinical Endocrinology & Diabetes. 109:S424-437.
87. Redlitz, A., Daum, G., and Sage, E. H. 1999. Angiostatin diminishes activation of the mitogen-activated protein kinases ERK-1 and ERK-2 in human dermal microvascular endothelial cells. J. Vasc. Res. 36:28-34.
88. Rohrer, B., and Stell, W. K. 1995. Localization of putative dopamine D2-like receptors in the chick retina, using in situ hybridization and immunocytochemistry. Brain Res. 695:110-116.
89. Sakharova, O. V., Taal, M. W., and Brenner, B. M. 2001. Pathogenesis of diabetic nephropathy: focus on transforming growth factor-beta and connective tissue growth factor. Current Opinion in Nephrology & Hypertension. 10:727-738.
90. Senger, D. R., Connolly, D. T., Van de Water, L., Feder, J., and Dvorak, H. F. 1990. Purification and NH2-terminal amino acid sequence of guinea pig tumor-secreted vascular permeability factor. Cancer Res. 50:1774-1778.
91. Senthil D, Choudhury G G, McLaurin C, Kasinath B S: Vascular endothelial growth factor induces protein synthesis in renal epithelial cells: a potential role in diabetic nephropathy.[see comment]. Kidney Int 64: 468-479, 2003.
92. Shestakova, M. V., Kochemasova, T. V., Gorelysheva, V. A., Osipova, T. V., Polosukhina, E. P., Baryshnikov, A., and Dedov, I I. 2002. [The role of adhesion molecules (ICAM-1 and E-selectin) in development of diabetic microangiopathies]. Terapevticheskii Arkhiv 74:24-27.
93. Shumway, J. T., and Gambert, S. R. 2002. Diabetic nephropathy-pathophysiology and management. International Urology and Nephrology 34:257-264.
94. Sim, B. K., MacDonald, N. J., and Gubish, E. R. 2000. Angiostatin and endostatin: endogenous inhibitors of tumor growth. Cancer & Metastasis Reviews 19:181-190.
95. Sima, J., Zhang, S. X., Shao, C., Fant, J., and Ma, J.-X. 2004. The effect of angiostatin on vascular leakage and VEGF expression in rat retina. FEBS Letters 564:19-23.
96. Siragy, H. M., Awad, A., Abadir, P., and Webb, R. 2003. The angiotensin II type 1 receptor mediates renal interstitial content of tumor necrosis factor-alpha in diabetic rats. Endocrinology 144:2229-2233.
97. Smith, L. E., Wesolowski, E., McLellan, A., Kostyk, S. K., D'Amato, R., Sullivan, R., and D'Amore, P. A. 1994. Oxygen-induced retinopathy in the mouse. Invest. Ophthalmol. Vis. Sci. 35:101-111.
98. Smith, L. E., Kopchick, J. J., Chen, W., Knapp, J., Kinose, F., Daley, D., Foley, E., Smith, R. G., and Schaeffer, J. M. 1997. Essential role of growth hormone in ischemia-induced retinal neovascularization. Science 276:1706-1709.
99. Spranger, J., Hammes, H. P., Preissner, K. T., Schatz, H., and Pfeiffer, A. F. 2000. Release of the angiogenesis inhibitor angiostatin in patients with proliferative diabetic retinopathy: association with retinal photocoagulation. Diabetologia 43:1404-1407.
100. Stellmach V, Crawford S E, Zhou W, Bouck N: Prevention of ischemia-induced retinopathy by the natural ocular antiangiogenic agent pigment epithelium-derived factor. Proc Natl Acad Sci USA 98: 2593-2597, 2001.
101. Tamaki, K., and Okuda, S. 2003. Role of TGF-beta in the progression of renal fibrosis. Contributions to Nephrology. 139:44-65.
102. Tarui, T., Miles, L. A., and Takada, Y. 2001. Specific interaction of angiostatin with integrin alpha(v)beta(3) in endothelial cells. J. Biol. Chem. 276:39562-39568.
103. Tombran-Tink J, Chader G G, Johnson L V: PEDF: a pigment epithelium-derived factor with potent neuronal differentiative activity. Exp Eye Res 53: 411-414, 1991.
104. Tombran-Tink J, Shivaram S M, Chader G J, Johnson L V, Bok D: Expression, secretion, and age-related downregulation of pigment epithelium-derived factor, a serpin with neurotrophic activity. J Neurosci 15: 4992-5003, 1995.
105. Tombran-Tink J, Mazuruk K, Rodriguez I R, Chung D, Linker T, Englander E, Chader G J: Organization, evolutionary conservation, expression and unusual Alu density of the human gene for pigment epithelium-derived factor, a unique neurotrophic serpin. Mol Vis 2: 11-11, 1996.
106. Tombran-Tink J, Barnstable C J: PEDF: a multifaceted neurotrophic factor. Nat Rev Neurosci 4: 628-636, 2003.
107. Tombran-Tink J, Barnstable C J: Therapeutic prospects for PEDF: more than a promising angiogenesis inhibitor. Trends Mol Med 9: 244-250, 2003.
108. Uehara H, Miyamoto M, Kato K, Ebihara Y, Kaneko H, Hashimoto H, Murakami Y, Hase R, Takahashi R, Mega S, Shichinohe T, Kawarada Y, Itoh T, Okushiba S, Kondo S, Katoh H: Expression of pigment epithelium-derived factor decreases liver metastasis and correlates with favorable prognosis for patients with ductal pancreatic adenocarcinoma. Cancer Res 64: 3533-3537, 2004.
109. Wada, T., Yokoyama, H., Matsushima, K., and Kobayashi, K. 2003. Monocyte chemoattractant protein-1: does it play a role in diabetic nephropathy? Nephrology Dialysis Transplantation 18:457-459.
110. Weston B S, Wahab N A, Mason R M: CTGF mediates TGF-beta-induced fibronectin matrix deposition by upregulating active alpha5beta1 integrin in human mesangial cells. J Am Soc Nephrol 14: 601-610, 2003.
111. Xu, Q., Qaum, T., and Adamis, A. P. 2001. Sensitive blood-retinal barrier breakdown quantitation using Evans blue. Invest. Ophthalmol. Vis. Sci. 42:789-794.
112. Yoshida, A., Ishiko, S., Kojima, M., and Ogasawara, H. 1993. Permeability of the blood-ocular barrier in adolescent and adult diabetic patients. Br. J. Ophthalmol. 77:158-161.
113. Zhang, D., Kaufman, P. L., Gao, G., Saunders, R. A., and Ma, J. X. 2001. Intravitreal injection of plasminogen kringle 5, an endogenous angiogenic inhibitor, arrests retinal neovascularization in rats. Diabetologia 44:757-765.
114. Zhang, S. X., Sima, J., Shao, C., Fant, J., Chen, Y., Rohrer, B., Gao, G. and Ma, J. (2004) Diabetologia 47, 124-131.
115. Zheng, F., Fornoni, A., Elliot, S. J., Guan, Y., Breyer, M. D., Striker, L. J., and Striker, G. E. 2002. Upregulation of type I collagen by TGF-beta in mesangial cells is blocked by PPARgamma activation. American Journal of Physiology—Renal Fluid & Electrolyte Physiology. 282:F639-648.

1. A method of inhibiting at least one of vascular leakage, inflammation and fibrosis in an animal having a disease or predisposition for vascular leakage, inflammation and fibrosis, comprising the step of:
administering to the animal a vascular leakage inhibiting amount of a composition, wherein at a substantially higher amount the composition is effective in inhibiting angiogenesis, and wherein the anti-angiogenic activity of the composition is separate from the vascular leakage inhibiting activity of the composition.

2. The method of claim 1 wherein the disease or predisposition is selected from the group consisting of diabetes, chronic inflammation, brain edema, arthritis, uvietis, macular edema, cancer, hyperglycemia, a kidney inflammatory disease, a disorder resulting in kidney fibrosis, a disorder of the kidney resulting in proteinuria, and combinations thereof.

3. The method of claim 1 wherein the effective amount of the composition capable of inhibiting at least one of vascular leakage, inflammation and fibrosis is at least 10-fold lower than the effective amount required to inhibit angiogenesis.

4. The method of claim 1 wherein the effective amount of the composition capable of inhibiting at least one of vascular leakage, inflammation and fibrosis is at least 50-fold lower than the effective amount required to inhibit angiogenesis.

5. The method of claim 1 wherein the effective amount of the composition capable of inhibiting at least one of vascular leakage, inflammation and fibrosis is at least 100-fold lower than the effective amount required to inhibit angiogenesis.

6. The method of claim 1 wherein the vascular leakage inhibiting amount of a composition substantially decreases overexpression of VEGF in at least one of retinas and kidneys of the animal.

7. The method of claim 1 wherein the vascular leakage inhibiting amount of a composition substantially decreases overexpression of TGF-β in at least one of retinas and kidneys of the animal.

8. The method of claim 1 wherein the vascular leakage inhibiting amount of a composition substantially decreases extracellular matrix overproduction in kidneys of the animal.

9. The method of claim 1 wherein the vascular leakage inhibiting amount of the composition substantially decreases overproduction of at least one inflammatory factor.

10. The method of claim 9 wherein the at least one inflammatory factor is MCP-1.

11. The method of claim 1 wherein the composition is a natural peptide that exhibits substantially no toxicity in the animal.

12. The method of claim 1 wherein the animal is a mammal.

13. The method of claim 1 wherein the animal is a human.

14. The method of claim 1 wherein the composition is selected from the group consisting of angiostatin, fragments of angiostatin, analogs or derivatives of angiostatin, pigment epithelium-derived factor, fragments of pigment epithelium-derived factor, analogs or derivatives of pigment epithelium-derived factor, SLED compounds and combinations thereof.

15. A method of inhibiting at least one of vascular leakage, inflammation and fibrosis, comprising the step of:
administering an effective amount of a composition capable of inhibiting at least one of vascular leakage, inflammation and fibrosis to an animal, in need thereof, wherein the composition capable of inhibiting at least one of vascular leakage, inflammation and fibrosis is selected from the group consisting of angiostatin, fragments of angiostatin, analogs or derivatives of angiostatin, pigment epithelium-derived factor, fragments of pigment epithelium-derived factor, analogs or derivatives of pigment epithelium-derived factor, SLED compounds and combinations thereof.

16. The method of claim 15 wherein the animal has a disease or a predisposition for a disease selected from the group consisting of diabetes, chronic inflammation, brain edema, arthritis, uvietis, macular edema, cancer, hyperglycemia, a kidney inflammatory disease, a disorder resulting in kidney fibrosis, a disorder of the kidney resulting in proteinuria, and combinations thereof.

17. The method of claim 15 wherein the composition capable of inhibiting at least one of vascular leakage, inflammation and fibrosis also exhibits anti-angiogenic properties.

18. The method of claim 17 wherein the effective amount of the composition capable of inhibiting at least one of vascular leakage, inflammation and fibrosis is a substantially lower amount than the effective amount of the composition required to inhibit angiogenesis.

19. The method of claim 18 wherein the effective amount of the composition capable of inhibiting at least one of vascular leakage, inflammation and fibrosis is at least 10-fold lower than the effective amount required to inhibit angiogenesis.

20. The method of claim 18 wherein the effective amount of the composition capable of inhibiting at least one of vascular leakage, inflammation and fibrosis is at least 50-fold lower than the effective amount required to inhibit angiogenesis.

21. The method of claim 18 wherein the effective amount of the composition capable of inhibiting at least one of vascular leakage, inflammation and fibrosis is at least 100-fold lower than the effective amount required to inhibit angiogenesis.

22. The method of claim 15 wherein the vascular leakage inhibiting amount of a composition substantially decreases overexpression of VEGF in at least one of retinas and kidneys of the animal.

23. The method of claim 15 wherein the vascular leakage inhibiting amount of a composition substantially decreases overexpression of TGF-β in at least one of retinas and kidneys of the animal.

24. The method of claim 15 wherein the vascular leakage inhibiting amount of a composition substantially decreases extracellular matrix overproduction in kidneys of the animal.

25. The method of claim 15 wherein the vascular leakage inhibiting amount of the composition substantially decreases overproduction of at least one inflammatory factor.

26. The method of claim 25 wherein the at least one inflammatory factor is MCP-1.

27. The method of claim 15 wherein the composition is a natural peptide that exhibits substantially no toxicity in the animal.

28. The method of claim 15 wherein the animal is a mammal.

29. The method of claim 15 wherein the animal is a human.

30. A composition comprising:
an activity that inhibits at least one of vascular leakage, inflammation and fibrosis;
an activity that inhibits angiogenesis; and
wherein a substantially higher amount of the composition must be administered to an animal for the composition to exhibit the inhibition of angiogenesis activity, whereas a substantially lower amount of the composition exhibits the activity that inhibits at least one of vascular leakage, inflammation and fibrosis when administered to an animal.

31. The composition of claim 30 wherein the amount of the composition capable of inhibiting at least one of vascular leakage, inflammation and fibrosis is at least 10-fold lower than the effective amount required to inhibit angiogenesis.

32. The composition of claim 30 wherein the amount of the composition capable of inhibiting at least one of vascular leakage, inflammation and fibrosis is at least 50-fold lower than the effective amount required to inhibit angiogenesis.

33. The composition of claim 30 wherein the effective amount of the composition capable of inhibiting at least one of vascular leakage, inflammation and fibrosis is at least 100-fold lower than the effective amount required to inhibit angiogenesis.

34. The composition of claim 30 wherein the composition is a natural peptide that exhibits substantially no toxicity in the animal.

35. The composition of claim 30 wherein the composition is selected from the group consisting of angiostatin, fragments of angiostatin, analogs or derivatives of angiostatin, pigment epithelium-derived factor, fragments of pigment epithelium-derived factor, analogs or derivatives of pigment epithelium-derived factor and combinations thereof.
